(19) United States
(12) Patent Application Publication
(10) Pub. No.: US 2008/0114172 A1
(43) Pub. Date: May 15, 2008

(54) Synthesis of Selected Stereoisomers of Certain Substituted Alcohols

(76) Inventor: Arthur E. Harms, Fairport, NY (US)

Correspondence Address: Bausch & Lomb Incorporated, One Bausch & Lomb Place, Rochester, NY, 14604-2701, US

(21) Appl. No.: 11/874,995

(22) Filed: Oct. 19, 2007

Related U.S. Application Data
(60) Provisional application No. 60/858,028, filed on Nov. 9, 2006.

(51) Int. Cl. C07D 405/12

(52) U.S. Cl. 546/143; 546/171

(57) ABSTRACT

A process for producing one selected stereoisomer of a substituted alcohol comprises reacting a stereoisomeric epoxide with an amine, a carboxylic acid, an amide, a sulfonyl, or a cyanide. The process avoids the production of a racemic mixture of stereoisomers of the prior art. Such a stereoisomeric substituted alcohol can be used for anti-inflammatory therapy.

### CROSS-REFERENCE

This application claims the benefit of Provisional Patent Application No. 60/858,028 filed Nov. 9, 2006 which is incorporated by reference herein.

### BACKGROUND OF THE INVENTION

The present invention relates to the synthesis of selected stereoisomers of certain substituted alcohols. In particular, the present invention relates to the selective synthesis of one of two possible stereoisomers of certain substituted alcohols.

The interface between the body and its environment is large, and thus presents many potential opportunities for invasion by environmental virulent pathogens. The outer tissues of the eye constitute parts of this interface, and thus, the eye and its surrounding tissues are also vulnerable to virulent microorganisms, the invasion and uncontrolled growth of which cause various types of ophthalmic infections, such as blepharitis, conjunctivitis, keratitis, or trachoma, which can result in serious impairment of vision if untreated. The common types of microorganisms causing ophthalmic infections are viruses, bacteria, and fungi. These microorganisms may directly invade the surface of the eye, or permeate into the globe of the eye through trauma or surgery, or transmit into the eye through the blood stream or lymphatic system as a consequence of a systemic disease. The microorganisms may attack any part of the eye structure, including the conjunctiva, the cornea, the uvea, the vitreous body, the retina, and the optic nerve. Ophthalmic infections can cause severe pain, swollen and red tissues in or around the eye, and blurred and decreased vision.

The body's innate cascade is activated soon after invasion by a foreign pathogen begins. Leukocytes (neutrophils, eosinophils, basophils, monocytes, and macrophages) are attracted to the site of infection in an attempt to eliminate the foreign pathogen through phagocytosis. Leukocytes and some affected tissue cells are activated by the pathogens to synthesize and release proinflammatory cytokines such as IL-1 β, IL-3, IL-5, IL-6, IL-8, TNF-α (tumor necrosis factor-α), GM-CSF (granulocyte-macrophage colony-stimulating factor), and MCP-1 (monocyte chemotactic protein-1). These released cytokines then further attract more immune cells to the infected site, amplifying the response of the immune system to defend the host against the foreign pathogen. For example, IL-8 and MCP-1 are potent chemoattractants for, and activators of, neutrophils and monocytes, respectively, while GM-CSF prolongs the survival of these cells and increases their response to other proinflammatory agonists. TNF-α can activate both types of cell and can stimulate further release of IL-8 and MCP-1 from them. IL-1 and TNF-α are potent chemoattractants for T and B lymphocytes, which are activated to produce antibodies against the foreign pathogen.

Although an inflammatory response is essential to clear pathogens from the site of infection, a prolonged or overactive inflammatory response can be damaging to the surrounding tissues. For example, inflammation causes the blood vessels at the infected site to dilate to increase blood flow to the site. As a result, these dilated vessels become leaky. After prolonged inflammation, the leaky vessels can produce serious edema in, and impair the proper functioning of, the surrounding tissues (see; e.g., V. W. M. van Hinsbergh, Arteriosclerosis, Thrombosis, and Vascular Biology, Vol. 17, 1018 (1997)). In addition, a continued dominating presence of macrophages at the injured site continues the production of toxins (such as reactive oxygen species) and matrix-degrading enzymes (such as matrix metalloproteinases) by these cells, which are injurious to both the pathogen and the host's tissues. Therefore, a prolonged or overactive inflammation should be controlled to limit the unintended damages to the body and to hasten the body's recovery process.

Glucocorticoids (also referred to herein as “corticosteroids”) represent one of the most effective clinical treatment for a range of inflammatory conditions, including acute inflammation. However, steroidal drugs can have side effects that threaten the overall health of the patient.

It is known that certain glucocorticoids have a greater potential for elevating intraocular pressure (“IOP”) than other compounds in this class. For example, it is known that prednisolone, which is a very potent ocular anti-inflammatory agent, has a greater tendency to elevate IOP than fluorometholone, which has moderate ocular anti-inflammatory activity. It is also known that the risk of IOP elevations associated with the topical ophthalmic use of glucocorticoids increases over time. In other words, the chronic (i.e., long-term) use of these agents increases the risk of significant IOP elevations. Unlike acute ocular inflammation associated with physical trauma or infection of the outer surface of the anterior portion of the eye, which requires short-term therapy on the order of a few weeks, infection and inflammation of the posterior portion of the eye can require treatment for extended periods of time, generally several months or more. This chronic use of corticosteroids significantly increases the risk of IOP elevations. In addition, use of corticosteroids is also known to increase the risk of cataract formation in a dose- and duration-dependent manner. Once cataracts develop, they may progress despite discontinuation of corticosteroid therapy.

Chronic administration of glucocorticoids also can lead to drug-induced osteoporosis by suppressing intestinal calcium absorption and inhibiting bone formation. Other adverse side effects of chronic administration of glucocorticoids include hypertension, hyperglycemia, hyperlipidemia (increased levels of triglycerides) and hypercholesterolemia (increased levels of cholesterol) because of the effects of these drugs on the body metabolic processes.

Therefore, there is a continued need to provide pharmaceutical compounds and compositions to treat, control, reduce, ameliorate, or prevent inflammation or infections and their inflammatory sequelae, which compounds and compositions cause a lower level of at least an adverse side effect than a composition comprising at least a prior-art glucocorticoid used to treat, reduce, or ameliorate the same conditions. Certain substituted alcohols have been disclosed to have anti-inflammatory properties similar to those of glucocorticoids, but with lower levels of some side effects (see; e.g., U.S. Pat. Nos. 6,897,224 and 7,109,212 and U.S. Patent Application Publication 2006/0116396). It is often found that one of the stereoisomers of these substituted alcohols has higher efficacy than the other stereoisomer. However, the prior-art syntheses of these substituted alcohols (as disclosed in these patents and patent application) typically yield a racemic mixture, which requires elaborate separation and increases the manufacturing cost. Therefore, it is very desirable to provide a method for producing only the selected stereoisomer of a desired substituted alcohol.

### SUMMARY

In general, the present invention provides a method for selectively producing a stereoisomer of a substituted alcohol that has a Formula Ia or Ib,

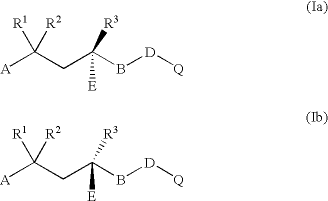

wherein A and Q are independently selected from the group consisting of unsubstituted and substituted aryl and heteroaryl groups, unsubstituted and substituted cycloalkyl and heterocycloalkyl groups, unsubstituted and substituted cycloalkenyl and heterocycloalkenyl groups, unsubstituted and substituted cycloalkynyl and heterocycloalkynyl groups, and unsubstituted and substituted heterocyclic groups; R¹ and R² are independently selected from the group consisting of hydrogen, unsubstituted C₁-C₁₅ (alternatively, C₁-C₁₀, or C₁-C₅, or C₁-C₃) linear or branched alkyl groups, substituted C₁-C₁₅ (alternatively, C₁-C₁₀, or C₁-C₅, or C₁-C₃) linear or branched alkyl groups, unsubstituted C₃-C₁₅ cycloalkyl groups, and substituted C₃-C₁₅ (alternatively, C₃-C₆, or C₃-C₅) cycloalkyl groups; R³ is selected from the group consisting of hydrogen, unsubstituted C₁-C₁₅ (alternatively, C₁-C₁₀, or C₁-C₅, or C₁-C₃) linear or branched alkyl groups, substituted C₁-C₁₅ (alternatively, C₁-C₁₀, or C₁-C₅, or C₁-C₃) linear or branched alkyl groups, unsubstituted C₃-C₁₅ (alternatively, C₃-C₆, or C₃-C₅) cycloalkyl and heterocycloalkyl groups, substituted C₃-C₁₅ (alternatively, C₃-C₆, or C₃-C₅) cycloalkyl and heterocycloalkyl groups, aryl groups, heteroaryl groups, and heterocyclylic groups; B comprises a methylene or substituted methylene group, wherein one or two substituents on the methylene group are independently C₁-C₅ alkyl (or alternatively, C₁-C₃ alkyl), hydroxy, halogen, amino, or oxo group; E is hydroxy; and D is —NH—, —NR′—, —OC(O)—, —C(O)NH—, —C(O)N(R′)—, —C(O)—, or —S—, wherein R′ comprises an unsubstituted or substituted C₁-C₁₅ (alternatively, C₁-C₁₀, or C₁-C₅, or C₁-C₃) linear or branched alkyl group; and wherein R¹ and R² together may form an unsubstituted or substituted C₃-C₁₅ cycloalkyl group. The method comprises reacting a compound having Formula IVa or IVb

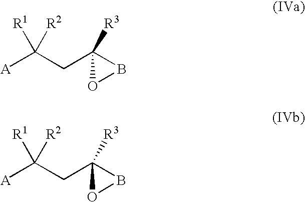

with a compound having a formula of Q-NH₂ (or Q-NHR′), Q-C(O)OH, Q-C(O)NH—R″ (or Q-C(O)N(R′)R″), or Q-SH, wherein R″ is hydrogen or a C₁-C₅ alkyl group (preferably, C₁-C₃ alkyl group).

Other features and advantages of the present invention will become apparent from the following detailed description and claims.

### DETAILED DESCRIPTION

Glucocorticoids (“GCs”) are among the most potent drugs used for the treatment of allergic and chronic inflammatory diseases or of inflammation resulting from infections. However, as mentioned above, long-term treatment with GCs is often associated with numerous adverse side effects, such as diabetes, osteoporosis, hypertension, glaucoma, or cataract. These side effects, like other physiological manifestations, are results of aberrant expression of genes responsible for such diseases. Research in the last decade has provided important insights into the molecular basis of GC-mediated actions on the expression of GC-responsive genes. GCs exert most of their genomic effects by binding to the cytoplasmic GC receptor (“GR”). The binding of GC to GR induces the translocation of the GC-GR complex to the cell nucleus where it modulates gene transcription either by a positive (transactivation) or negative (transrepression) mode of regulation. There has been growing evidence that both beneficial and undesirable effects of GC treatment are the results of undifferentiated levels of expression of these two mechanisms; in other words, they proceed at similar levels of effectiveness. Although it has not yet been possible to ascertain the most critical aspects of action of GCs in chronic inflammatory diseases, there has been evidence that it is likely that the inhibitory effects of GCs on cytokine synthesis are of particular importance. GCs inhibit the transcription, through the transrepression mechanism, of several cytokines that are relevant in inflammatory diseases, including IL-1β (interleukin-1β), IL-2, IL-3, IL-6, IL-11, TNF-α (tumor necrosis factor-α), GM-CSF (granulocyte-macrophage colony-stimulating factor), and chemokines that attract inflammatory cells to the site of inflammation, including IL-8, RANTES, MCP-1 (monocyte chemotactic protein-1), MCP-3, MCP-4, MIP-1α (macrophage-inflammatory protein-1α), and eotaxin. P. J. Barnes, Clin. Sci., Vol. 94, 557-572 (1998). On the other hand, there is persuasive evidence that the synthesis of IκBα, which are proteins having inhibitory effects on the NF-κB proinflammatory transcription factors, is increased by GCs. These proinflammatory transcription factors regulate the expression of genes that code for many inflammatory proteins, such as cytokines, inflammatory enzymes, adhesion molecules, and inflammatory receptors. S. Wissink et al., Mol. Endocrinol., Vol. 12, No. 3, 354-363 (1998); P. J. Barnes and M. Karin, New Engl. J. Med., Vol. 336, 1066-1077 (1997). Thus, both the transrepression and transactivation functions of GCs directed to different genes produce the beneficial effect of inflammatory inhibition. On the other hand, steroid-induced diabetes and glaucoma appear to be produced by the transactivation action of GCs on genes responsible for these diseases. H. Schäcke et al., Pharmacol. Ther., Vol. 96, 23-43 (2002). Thus, while the transactivation of certain genes by GCs produces beneficial effects, the transactivation of other genes by the same GCs can produce undesired side effects, one of which is glaucoma. Therefore, GCs would not be employed to treat or prevent glaucoma or its progression. Consequently, it is very desirable to provide pharmaceutical compounds and compositions that produce differentiated levels of transactivation and transrepression activity on GC-responsive genes such that undesired side effects are not produced or at least are minimized.

In certain aspects, a compound that produces differentiated levels of transactivation and transrepression activity on GC-responsive genes such that undesired side effects are not produced or at least are minimized can satisfy some unmet needs for therapies that heretofore have relied on glucocorticoids. Such a compound, termed herein a dissociated glucocorticoid receptor agonist (“DIGRA”), is capable of binding to the glucocorticoid receptor (which is a polypeptide) and, upon binding, is capable of producing differentiated levels of transrepression and transactivation of gene expression. A compound that binds to a polypeptide is sometimes herein referred to as a ligand.

As used herein, the term “alkyl” or “alkyl group” means a linear- or branched-chain saturated aliphatic hydrocarbon monovalent group, which may be unsubstituted or substituted. The group may be partially or completely substituted with halogen atoms (F, Cl, Br, or I). Non-limiting examples of alkyl groups include methyl, ethyl, n-propyl, 1-methylethyl(isopropyl), n-butyl, n-pentyl, 1,1-dimethylethyl (t-butyl), and the like. It may be abbreviated as “Alk”.

As used herein, the term “alkenyl” or “alkenyl group” means a linear- or branched-chain aliphatic hydrocarbon monovalent radical containing at least one carbon-carbon double bond. This term is exemplified by groups such as ethenyl, propenyl, n-butenyl, isobutenyl, 3-methylbut-2-enyl, n-pentenyl, heptenyl, octenyl, decenyl, and the like.

As used herein, the term “alkynyl” or “alkynyl group” means a linear- or branched-chain aliphatic hydrocarbon monovalent radical containing at least one carbon-carbon triple bond. This term is exemplified by groups such as ethynyl, propynyl, n-butynyl, 2-butynyl, 3-methylbutynyl, n-pentynyl, heptynyl, octynyl, decynyl, and the like.

As used herein, the term “alkylene” or “alkylene group” means a linear- or branched-chain saturated aliphatic hydrocarbon divalent radical having the specified number of carbon atoms. This term is exemplified by groups such as methylene, ethylene, propylene, n-butylene, and the like, and may alternatively and equivalently be denoted herein as “-(alkyl)-”.

The term “alkenylene” or “alkenylene group” means a linear- or branched-chain aliphatic hydrocarbon divalent radical having the specified number of carbon atoms and at least one carbon-carbon double bond. This term is exemplified by groups such as ethenylene, propenylene, n-butenylene, and the like, and may alternatively and equivalently be denoted herein as “-(alkylenyl)-”.

The term “alkynylene” or “alkynylene group” means a linear- or branched-chain aliphatic hydrocarbon divalent radical containing at least one carbon-carbon triple bond. This term is exemplified by groups such as ethynylene, propynylene, n-butenylene, 2-butenylene, 3-methylbutynylene, n-pentenylene, heptenylene, octynylene, decynylene, and the like, and may alternatively and equivalently be denoted herein as “-(alkynyl)-”.

As used herein, the term “aryl” or “aryl group” means an aromatic carbocyclic monovalent or divalent radical of from 5 to 16 carbon atoms having a single ring (e.g., phenyl or phenylene), multiple condensed rings (e.g., naphthyl or anthranyl), or multiple bridged rings (e.g., biphenyl). Unless otherwise specified, the aryl ring may be attached at any suitable carbon atom which results in a stable structure and, if substituted, may be substituted at any suitable carbon atom which results in a stable structure. In some embodiments, the aryl group comprises from 5 to 14 carbon atoms. In some other embodiments, the aryl group comprises from 5 to 10 carbon atoms. Non-limiting examples of aryl groups include phenyl, naphthyl, anthryl, phenanthryl, indanyl, indenyl, biphenyl, and the like. It may be abbreviated as “Ar”.

The term “heteroaryl” or “heteroaryl group” means a stable aromatic 5- to 16-membered, monocyclic or polycyclic monovalent or divalent radical, which may comprise one or more fused or bridged ring(s), preferably a 5- to 7-membered monocyclic or 7- to 10-membered bicyclic radical, having from one to four heteroatoms in the ring(s) independently selected from nitrogen, oxygen, and sulfur, wherein any sulfur heteroatoms may optionally be oxidized and any nitrogen heteroatom may optionally be oxidized or be quaternized. Unless otherwise specified, the heteroaryl ring may be attached at any suitable heteroatom or carbon atom which results in a stable structure and, if substituted, may be substituted at any suitable heteroatom or carbon atom which results in a stable structure. Non-limiting examples of heteroaryls include furanyl, thienyl, pyrrolyl, oxazolyl, thiazolyl, imidazolyl, pyrazolyl, isoxazolyl, isothiazolyl, oxadiazolyl, triazolyl, tetrazolyl, thiadiazolyl, pyridinyl, pyridazinyl, pyrimidinyl, pyrazinyl, triazinyl, indolizinyl, azaindolizinyl, indolyl, azaindolyl, diazaindolyl, dihydroindolyl, dihydroazaindoyl, isoindolyl, azaisoindolyl, benzofuranyl, furanopyridinyl, furanopyrimidinyl, furanopyrazinyl, furanopyridazinyl, dihydrobenzofuranyl, dihydrofuranopyridinyl, dihydrofuranopyrimidinyl, benzothienyl, thienopyridinyl, thienopyrimidinyl, thienopyrazinyl, thienopyridazinyl, dihydrobenzothienyl, dihydrothienopyridinyl, dihydrothienopyrimidinyl, indazolyl, azaindazolyl, diazaindazolyl, benzimidazolyl, imidazopyridinyl, benzthiazolyl, thiazolopyridinyl, thiazolopyrimidinyl, benzoxazolyl, benzoxazinyl, benzoxazinonyl, oxazolopyridinyl, oxazolopyrimidinyl, benzisoxazolyl, purinyl, chromanyl, azachromanyl, quinolizinyl, quinolinyl, dihydroquinolinyl, tetrahydroquinolinyl, isoquinolinyl, dihydroisoquinolinyl, tetrahydroisoquinolinyl, cinnolinyl, azacinnolinyl, phthalazinyl, azaphthalazinyl, quinazolinyl, azaquinazolinyl, quinoxalinyl, azaquinoxalinyl, naphthyridinyl, dihydronaphthyridinyl, tetrahydronaphthyridinyl, pteridinyl, carbazolyl, acridinyl, phenazinyl, phenothiazinyl, and phenoxazinyl, and the like.

The term “heterocycle”, “heterocycle group”, “heterocyclyl”, “heterocyclyl group”, “heterocyclic”, or “heterocyclic group” means a stable non-aromatic 5- to 16-membered monocyclic or polycyclic, monovalent or divalent, ring which may comprise one or more fused or bridged ring(s), preferably a 5- to 7-membered monocyclic or 7- to 10-membered bicyclic ring, having from one to three heteroatoms in at least one ring independently selected from nitrogen, oxygen, and sulfur, wherein any sulfur heteroatoms may optionally be oxidized and any nitrogen heteroatom may optionally be oxidized or be quaternized. As used herein, a heterocyclyl group excludes heterocycloalkyl, heterocycloalkenyl, and heterocycloalkynyl groups. Unless otherwise specified, the heterocyclyl ring may be attached at any suitable heteroatom or carbon atom which results in a stable structure and, if substituted, may be substituted at any suitable heteroatom or carbon atom which results in a stable structure. Non-limiting examples of heterocycles include pyrrolinyl, pyrrolidinyl, pyrazolinyl, pyrazolidinyl, piperidinyl, morpholinyl, thiomorpholinyl, piperazinyl, tetrahydropyranyl, tetrahydrothiopyranyl, tetrahydrofuranyl, hexahydropyrimidinyl, hexahydropyridazinyl, and the like.

The term “cycloalkyl” or “cycloalkyl group” means a stable aliphatic saturated 3- to 15-membered monocyclic or polycyclic monovalent radical consisting solely of carbon and hydrogen atoms which may comprise one or more fused or bridged ring(s), preferably a 5- to 7-membered monocyclic or 7- to 10-membered bicyclic ring. Unless otherwise specified, the cycloalkyl ring may be attached at any carbon atom which results in a stable structure and, if substituted, may be substituted at any suitable carbon atom which results in a stable structure. Exemplary cycloalkyl groups include cyclopropyl, cyclobutyl, cyclopentyl, cyclohexyl, cycloheptyl, cyclooctyl, cyclononyl, cyclodecyl, norbornyl, adamantyl, tetrahydronaphthyl (tetralin), 1-decalinyl, bicyclo[2.2.2]octanyl, 1-methylcyclopropyl, 2-methylcyclopentyl, 2-methylcyclooctyl, and the like.

The term “cycloalkenyl” or “cycloalkenyl group” means a stable aliphatic 5- to 15-membered monocyclic or polycyclic monovalent radical having at least one carbon-carbon double bond and consisting solely of carbon and hydrogen atoms which may comprise one or more fused or bridged ring(s), preferably a 5- to 7-membered monocyclic or 7- to 10-membered bicyclic ring. Unless otherwise specified, the cycloalkenyl ring may be attached at any carbon atom which results in a stable structure and, if substituted, may be substituted at any suitable carbon atom which results in a stable structure. Exemplary cycloalkenyl groups include cyclopentenyl, cyclohexenyl, cycloheptenyl, cyclooctenyl, cyclononenyl, cyclodecenyl, norbornenyl, 2-methylcyclopentanyl, 2-methylcyclooctenyl, and the like.

The term “cycloalkynyl” or “cycloalkynyl group” means a stable aliphatic 8- to 15-membered monocyclic or polycyclic monovalent radical having at least one carbon-carbon triple bond and consisting solely of carbon and hydrogen atoms which may comprise one or more fused or bridged ring(s), preferably a 8- to 10-membered monocyclic or 12- to 15-membered bicyclic ring. Unless otherwise specified, the cycloalkynyl ring may be attached at any carbon atom which results in a stable structure and, if substituted, may be substituted at any suitable carbon atom which results in a stable structure. Exemplary cycloalkynyl groups include cyclooctynyl, cyclononenyl, cyclodecenyl, 2-methylcyclooctynyl, and the like.

The term “carbocycle” or “carbocyclic group” means a stable aliphatic 3- to 15-membered monocyclic or polycyclic monovalent or divalent radical consisting solely of carbon and hydrogen atoms which may comprise one or more fused or bridged rings, preferably a 5- to 7-membered monocyclic or 7- to 10-membered bicyclic ring. Unless otherwise specified, the carbocycle may be attached at any carbon atom which results in a stable structure and, if substituted, may be substituted at any suitable carbon atom which results in a stable structure. The term comprises cycloalkyl (including spiro cycloalkyl), cycloalkylene, cycloalkenyl, cycloalkenylene, cycloalkynyl, and cycloalkenylene, and the like.

The terms “heterocycloalkyl”, “heterocycloalkenyl”, and “heterocycloalkynyl” mean cycloalkyl, cycloalkenyl, and cycloalkynyl group, respectively, having at least a heteroatom in at least one ring, respectively.

In general, the present invention provides a method for selectively producing a stereoisomeric compound having Formula Ia or Ib,

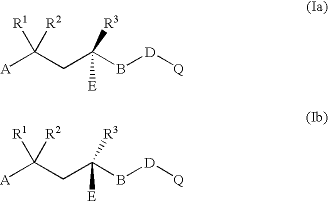

wherein A and Q are independently selected from the group consisting of unsubstituted and substituted aryl and heteroaryl groups, unsubstituted and substituted cycloalkyl and heterocycloalkyl groups, unsubstituted and substituted cycloalkenyl and heterocycloalkenyl groups, unsubstituted and substituted cycloalkynyl and heterocycloalkynyl groups, and unsubstituted and substituted heterocyclic groups; R¹ and R² are independently selected from the group consisting of hydrogen, unsubstituted C₁-C₁₅ (alternatively, C₁-C₁₀, or C₁-C₅, or C₁-C₃) linear or branched alkyl groups, substituted C₁-C₁₅ (alternatively, C₁-C₁₀, or C₁-C₅, or C₁-C₃) linear or branched alkyl groups, unsubstituted C₃-C₁₅ cycloalkyl groups, and substituted C₃-C₁₅ (alternatively, C₃-C₆, or C₃-C₅) cycloalkyl groups; R³ is selected from the group consisting of hydrogen, unsubstituted C₁-C₁₅ (alternatively, C₁-C₁₀, or C₁-C₅, or C₁-C₃) linear or branched alkyl groups, substituted C₁-C₁₅ (alternatively, C₁-C₁₀, or C₁-C₅, or C₁-C₃) linear or branched alkyl groups, unsubstituted C₃-C₁₅ (alternatively, C₃-C₆, or C₃-C₅) cycloalkyl and heterocycloalkyl groups, substituted C₃-C₁₅ (alternatively, C₃-C₆, or C₃-C₅) cycloalkyl and heterocycloalkyl groups, aryl groups, heteroaryl groups, and heterocyclylic groups; B comprises a methylene or substituted methylene group, wherein one or two substituents on the methylene group are independently C₁-C₅ alkyl (or alternatively, C₁-C₃ alkyl), hydroxy, halogen, amino, or oxo group; E is hydroxy; and D is —NH—, —NR′—, —OC(O)—, —C(O)NH—, —C(O)N(R′)—, —C(O—, or —S—, wherein R′ comprises an unsubstituted or substituted C₁-C₁₅ (alternatively, C₁-C₁₀, or C₁-C₅, or C₁-C₃) linear or branched alkyl group; and wherein R¹ and R² together may form an unsubstituted or substituted C₃-C₁₅ cycloalkyl group. The method comprises reacting a compound having Formula IVa or IVb

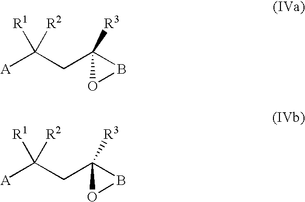

with a compound having a formula of Q-NH₂ (or Q-NHR′), Q-C(O)OH, Q-C(O)NH—R″ (or Q-C(O)N(R′)R″), or Q-SH wherein R″ is hydrogen or a C₁-C₅ alkyl group (preferably, C₁-C₃ alkyl group).

In one embodiment, B is the methylene group.

In another embodiment, A and Q are independently selected from the group consisting of aryl and heteroaryl groups substituted with at least a halogen atom, cyano group, hydroxy group, or C₁-C₁₀ alkoxy group (alternatively, C₁-C₅ alkoxy group, or C₁-C₃ alkoxy group); R¹, R², and R³ are independently selected from the group consisting of unsubstituted and substituted C₁-C₅ alkyl groups (preferably, C₁-C₃ alkyl groups); B is a methylene group; D is the —NH— or —NR′— group, wherein R′ is a C₁-C₅ alkyl group (preferably, C₁-C₃ alkyl group); and E is the hydroxy group.

In still another embodiment, A comprises a dihydrobenzofuranyl group substituted with a halogen atom; Q comprises a quinolinyl or isoquinolinyl group substituted with a C₁-C₁₀ alkyl group; R¹ and R² are independently selected from the group consisting of unsubstituted and substituted C₁-C₅ alkyl groups (preferably, C₁-C₃ alkyl groups); B is a methylene group; D is the —NH— group; E is the hydroxy group; and R³ comprises a completely halogenated C₁-C₁₀ alkyl group (preferably, completely halogenated C₁-C₅ alkyl group; more preferably, completely halogenated C₁-C₃ alkyl group).

In yet another embodiment, A comprises a dihydrobenzofuranyl group substituted with a fluorine atom; Q comprises a quinolinyl or isoquinolinyl group substituted with a methyl group; R¹ and R² are independently selected from the group consisting of unsubstituted and substituted C₁-C₅ alkyl groups; B is a methylene group; D is the —NH— group; E is the hydroxy group; and R³ comprises a trifluoromethyl group.

Compounds having Formula Ia or Ib are useful as a dissociated glucocorticoid receptor agonist (“DIGRA”).

In still another aspect, the present invention provides a method for producing stereoisomeric DIGRA compounds having Formula Ia, IIb, IIc, or IId,

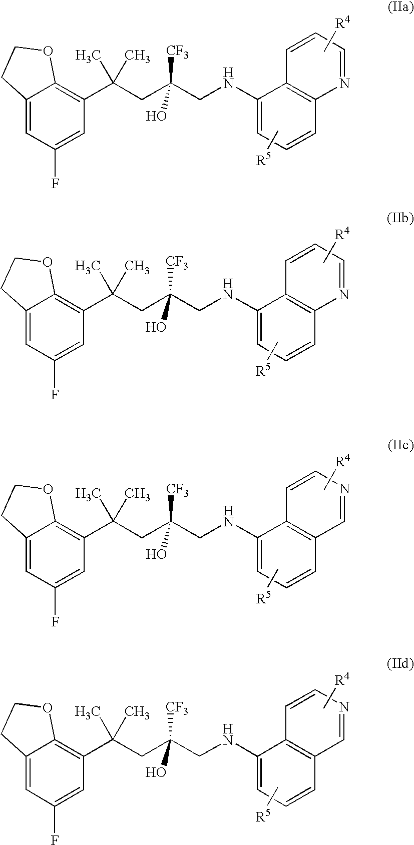

wherein R⁴ and R⁵ are independently selected from the group consisting of hydrogen, halogen, cyano, hydroxy, C₁-C₁₀ (alternatively, C₁-C₅ or C₁-C₃) alkoxy groups, unsubstituted C₁-C₁₀ (alternatively, C₁-C₅ or C₁-C₃) linear or branched alkyl groups, substituted C₁-C₁₀ (alternatively, C₁-C₅ or C₁-C₃) linear or branched alkyl groups, unsubstituted C₃-C₁₀ (alternatively, C₃-C₆ or C₃-C₅) cyclic alkyl groups, and substituted C₃-C₁₀ (alternatively, C₃-C₆ or C₃-C₅) cyclic alkyl groups.

In still another aspect, the present invention provides a method for producing stereoisomeric DIGRA compounds having Formula IIIa, IlIb, IIIc, or IIId.

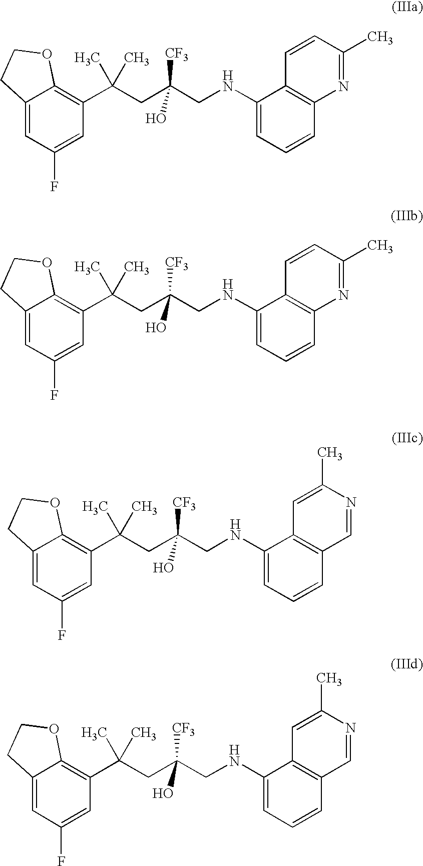

In still another aspect, the present invention provides a stereoisomeric compound having Formula Ia, Ib, Ia, IIb, IIIa, or IIIb and a method for their production, whence a prodrug, a pharmaceutically acceptable salt, or a pharmaceutically acceptable ester of such a stereoisomeric compound may be prepared.

Non-limiting examples of compounds having Formula Ia or Ib that may be produced by a method of the present invention include 5-[4-(5-fluoro-2,3-dihydrobenzofuran-7-yl)-2-hydroxy-4-methyl-2-trifluoromethyl-pentylamino]-2-methylquinoline, 5-[4-(5-fluoro-2,3-dihydrobenzofuran-7-yl)-2-hydroxy-4-methyl-2-trifluoromethyl-pentylamino]-1-methylisoquinolin, 5-[4-(5-fluoro-2,3-dihydrobenzofuran-7-yl)-2-hydroxy-4-methyl-2-trifluoromethyl-pentylamino]isoquinol-1(2H)-one, 5-[4-(5-fluoro-2,3-dihydrobenzofuran-7-yl)-2-hydroxy-4-methyl-2-trifluoromethyl-pentylamino]-2,6-dimethylquinoline, 5-[4-(5-fluoro-2,3-dihydrobenzofuran-7-yl)-2-hydroxy-4-methyl-2-trifluoromethyl-pentylamino]-6-chloro-2-methylquinoline, 5-[4-(5-fluoro-2,3-dihydrobenzofuran-7-yl)-2-hydroxy-4-methyl-2-trifluoromethyl-pentylamino]isoquinoline, 5-[4-(5-fluoro-2,3-dihydrobenzofuran-7-yl)-2-hydroxy-4-methyl-2-trifluoromethyl-pentylamino]quinoline, 5-[4-(2,3-dihydro-5-fluoro-7-benzofuranyl)-2-hydroxy-4-methyl-2-trifluoromethyl-pentylamino]quinolin-2-[1H]-one, 6-fluoro-5-[4-(5-fluoro-2,3-dihydrobenzofuran-7-yl)-2-hydroxy-4-methyl-2-trifluoromethyl-pentylamino]-2-methylquinoline, 8-fluoro-5-[4-(5-fluoro-2,3-dihydrobenzofuran-7-yl)-2-hydroxy-4-methyl-2-trifluoromethyl-pentylamino]-2-methylquinoline, 5-[4-(5-fluoro-2,3-dihydrobenzofuran-7-yl)-2-hydroxy-4-methyl-2-trifluoromethyl-pentylamino]-2-methylisoquinol-1-[2H]-one, and enantiomers thereof.

In yet another embodiment, the present invention provide a method for producing a stereoisomeric DIGRA compound having Formula Ia or Ib, wherein

(a) A is an aryl or heteroaryl group optionally independently substituted with one to three substituent groups, which are independently selected from the group consisting of C₁-C₅ alkyl, C₂-C₅ alkenyl, C₂-C₅ alkynyl, C₁-C₃ alkanoyl, C₃-C₈ cycloalkyl, heterocyclyl, aryl, heteroaryl, C₁-C₅ alkoxy, C₂-C₅ alkenyloxy, C₂-C₅ alkynyloxy, aryloxy, acyl, C₁-C₅ alkoxycarbonyl, aroyl, aminocarbonyl, alkylaminocarbonyl, dialkylaminocarbonyl, aminocarbonyloxy, C₁-C₅ alkylaminocarbonyloxy, C₁-C₅ dialkylaminocarbonyloxy, C₁-C₅ alkanoylamino, C₁-C₅ alkoxycarbonylamino, C₁-C₅ alkylsulfonylamino, aminosulfonyl, C₁-C₅ alkylaminosulfonyl, C₁-C₅ dialkylaminosulfonyl, halogen, hydroxy, carboxy, cyano, trifluoromethyl, trifluoromethoxy, nitro, amino wherein the nitrogen atom is optionally independently mono- or di-substituted by C₁-C₅ alkyl or aryl, ureido wherein either nitrogen atom is optionally independently substituted with C₁-C₅ alkyl, C₁-C₅ alkylthio wherein the sulfur atom is optionally oxidized to a sulfoxide or sulfone;

(b) R¹ and R² are each independently hydrogen or C₁-C₅ alkyl;

(c) R³ is the trifluoromethyl group;

(d) B is a methylene or substituted methylene group, wherein a substituent group of B is independently C₁-C₃ alkyl, hydroxy, halogen, amino, or oxo;

(e) D is —NH—, —NR′—, —OC(O)—, —C(O)NH—, —C(O)N(R′)—, —C(O)—, or —S—, R′ comprises an unsubstituted or substituted C₁-C₁₅ (alternatively, C₁-C₁₀, or C₁-C₅, or C₁-C₃) linear or branched alkyl group;

(f) E is the hydroxy group; and

(g) Q is an azaindolyl group optionally independently substituted with one to three substituent groups, wherein each substituent group of Q is independently C₁-C₅ alkyl, C₂-C₅ alkenyl, C₂-C₅ alkynyl, C₃-C₈ cycloalkyl, heterocyclyl, aryl, heteroaryl, C₁-C₅ alkoxy, C₂-C₅ alkenyloxy, C₂-C₅ alkynyloxy, aryloxy, acyl, C₁-C₅ alkoxycarbonyl, C₁-C₅ alkanoyloxy, aminocarbonyl, alkylaminocarbonyl, dialkylaminocarbonyl, aminocarbonyloxy, C₁-C₅ alkylaminocarbonyloxy, C₁-C₅ dialkylaminocarbonyloxy, C₁-C₅ alkanoylamino, C₁-C₅ alkoxycarbonylamino, C₁-C₅ alkylsulfonylamino, aminosulfonyl, C₁-C₅ alkylaminosulfonyl, C₁-C₅ dialkylaminosulfonyl, halogen, hydroxy, carboxy, cyano, trifluoromethyl, trifluoromethoxy, trifluoromethylthio, nitro, or amino wherein the nitrogen atom is optionally independently mono- or di-substituted by C₁-C₅ alkyl, ureido wherein either nitrogen atom is optionally independently substituted with C₁-C₅ alkyl, C₁-C₅ alkylthio wherein the sulfur atom is optionally oxidized to a sulfoxide or sulfone, wherein each substituent group of Q is optionally independently substituted with one to three substituent groups selected from the group consisting of C₁-C₃ alkyl, C₁-C₃ alkoxy, halogen, hydroxy, oxo, cyano, amino, and trifluoromethyl.

In certain embodiments, D is —NH— or —NR′—, wherein R′ comprises an unsubstituted or substituted C₁-C₁₅ (alternatively, C₁-C₁₀, or C₁-C₅, or C₁-C₃) linear or branched alkyl group.

Non-limiting examples of these compounds include 1,1,1-trifluoro-4-(5-fluoro-2-methoxyphenyl)-4-methyl-2-(((1H-pyrrolo[2,3-c]pyridin-2-yl)methylamino)methyl)pentan-2-ol; 1,1,1-trifluoro-4-(5-fluoro-2-methoxyphenyl)-4-methyl-2-(((1H-pyrrolo[3,2-c]pyridin-2-yl)methylamino)methyl)pentan-2-ol; 1,1,1-trifluoro-4-methyl-4-phenyl-2-(((1H-pyrrolo[2,3-c]pyridin-2-yl)methylamino)methyl)pentan-2-ol; 1,1,1-trifluoro-4-(4-fluoro-2-methoxyphenyl)-4-methyl-2-((1H-pyrrolo[2,3-c]pyridin-2-yl)methylamino)methyl)pentan-2-ol; 1,1,1-trifluoro-4-methyl-4-phenyl-2-(((1H-pyrrolo[3,2-c]pyridin-2-yl)methylamino)methyl)pentan-2-ol; 1,1,1-trifluoro-4-(4-fluoro-2-methoxyphenyl)-4-methyl-2-(((1H-pyrrolo[3,2-c]pyridin-2-yl)methylamino)methyl)pentan-2-ol; and 1,1,1-trifluoro-4-(5-fluoro-2-methoxyphenyl)-4-methyl-2-(((3-methyl-1H-pyrrolo[2,3-c]pyridin-2-yl)methylamino)methyl)pentan-2-ol.

In still another embodiment, the present invention provide a method for producing a stereoisomeric DIGRA compound having Formula Ia or Ib, wherein

(a) A is an aryl or heteroaryl group, each optionally independently substituted with one to three substituent groups, which are independently selected from the group consisting of C₁-C₅ alkyl, C₂-C₅ alkenyl, C₂-C₅ alkynyl, C₁-C₃ alkanoyl, C₃-C₈ cycloalkyl, heterocyclyl, aryl, heteroaryl, C₁-C₅ alkoxy, C₂-C₅ alkenyloxy, C₂-C₅ alkynyloxy, aryloxy, acyl, C₁-C₅ alkoxycarbonyl, aroyl, aminocarbonyl, alkylaminocarbonyl, dialkylaminocarbonyl, aminocarbonyloxy, C₁-C₅ alkylaminocarbonyloxy, C₁-C₅ dialkylaminocarbonyloxy, C₁-C₅ alkanoylamino, C₁-C₅ alkoxycarbonylamino, C₁-C₅ alkylsulfonylamino, aminosulfonyl, C₁-C₅ alkylaminosulfonyl, C₁-C₅ dialkylaminosulfonyl, halogen, hydroxy, carboxy, cyano, trifluoromethyl, trifluoromethoxy, nitro, amino wherein the nitrogen atom is optionally independently mono- or di-substituted by C₁-C₅ alkyl or aryl, ureido wherein either nitrogen atom is optionally independently substituted with C₁-C₅ alkyl, C₁-C₅ alkylthio wherein the sulfur atom is optionally oxidized to a sulfoxide or sulfone;

(b) R¹ and R² are each independently hydrogen or C₁-C₅ alkyl, or R¹ and R² together with the carbon atom they are commonly attached to form a C₃-C₈ spiro cycloalkyl ring;

(c) B is a methylene or substituted methylene group, wherein one or two substituents on the methylene group is C₁-C₅ alkyl (or alternatively, C₁-C₃ alkyl), hydroxy, amino, or oxo group;

(d) R³ is a carbocycle, heterocyclyl, aryl, heteroaryl, carbocycle-C₁-C₈ alkyl, aryl-C₁-C₈ alkyl, aryl-C₁-C₈ haloalkyl, heterocyclyl-C₁-C₈ alkyl, heteroaryl-C₁-C₈ alkyl, carbocycle-C₂-C₈ alkenyl, aryl-C₂-C₈ alkenyl, heterocyclyl-C₂-C₈ alkenyl, or heteroaryl-C₂-C₈ alkenyl, each optionally independently substituted with one to three substituent groups;

(e) D is —NH—, —NR′—, —OC(O)—, —C(O)NH—, —C(O)N(R′)—, —C(O), or —S— group, wherein R′ comprises an unsubstituted or substituted C₁-C₁₅ (alternatively, C₁-C₁₀, or C₁-C₅, or C₁-C₃) linear or branched alkyl group;

(f) E is the hydroxy group; and

(g) Q comprises a methylated benzoxazinone.

Non-limiting examples of these compounds include 6-[2-benzyl-4-(5-fluoro-2-methoxyphenyl)-2-hydroxy-4-methylpentylamino]-(4-methyl-1-oxo-1H-benzo[d[1,2]oxazine); 7-[2-benzyl-4-(5-fluoro-2-hydroxyphenyl)-2-hydroxy-4-methylpentylamino]-(4-methyl-1-oxo-1H-benzo[d][1,2]oxazine); 6-[2-cyclohexylmethyl-4-(5-fluoro-2-methoxyphenyl)-2-hydroxy-4-methylpentylamino]-(4-methyl-1-oxo-1H-benzo[d][1,2]oxazine); 6-[2-cyclohexylmethyl-4-(5-fluoro-2-hydroxyphenyl)-2-hydroxy-4-methylpentylamino]-(4-methyl-1-oxo-1H-benzo[d][1,2]oxazine); 5-benzyl-5-methyl-3-hydroxy-3-trifluoromethyl-hexanoic acid-(4-methyl-1-oxo-1H-benzo[d][1,2]oxazin-6-yl)amide; and 5-(2-methoxyphenyl)-3-cyclohexylmethyl-3-hydroxy-5-methylhexanoic acid-(4-methyl-1-oxo-1H-benzo[d][1,2]oxazin-6-yl)amide.

In still another embodiment, the present invention provide a method for producing a stereoisomeric DIGRA compound having Formula Ia or Ib, wherein

(a) A is an aryl or heteroaryl group, each optionally independently substituted with one to three substituent groups, which are independently selected from the group consisting of C₁-C₅ alkyl, C₂-C₅ alkenyl, C₂-C₅ alkynyl, C₁-C₃ alkanoyl, C₃-C₈ cycloalkyl, heterocyclyl, aryl, heteroaryl, C₁-C₅ alkoxy, C₂-C₅ alkenyloxy, C₂-C₅ alkynyloxy, aryloxy, acyl, C₁-C₅ alkoxycarbonyl, aroyl, aminocarbonyl, alkylaminocarbonyl, dialkylaminocarbonyl, aminocarbonyloxy, C₁-C₅ alkylaminocarbonyloxy, C₁-C₅ dialkylaminocarbonyloxy, C₁-C₅ alkanoylamino, C₁-C₅ alkoxycarbonylamino, C₁-C₅ alkylsulfonylamino, aminosulfonyl, C₁-C₅ alkylaminosulfonyl, C₁-C₅ dialkylaminosulfonyl, halogen, hydroxy, carboxy, cyano, trifluoromethyl, trifluoromethoxy, nitro, amino wherein the nitrogen atom is optionally independently mono- or di-substituted by C₁-C₅ alkyl or aryl, ureido wherein either nitrogen atom is optionally independently substituted with C₁-C₅ alkyl, C₁-C₅ alkylthio wherein the sulfur atom is optionally oxidized to a sulfoxide or sulfone;

(b) R¹ and R² are each independently hydrogen or C₁-C₅ alkyl, or R¹ and R² together with the carbon atom they are commonly attached to form a C₃-C₈ spiro cycloalkyl ring;

(c) R³ is C₁-C₁₀ alkyl or substituted C₁-C₁₀ alkyl group (in certain embodiments, R³ is a partially or completely halogenated C₁-C₁₀ alkyl group, and in certain other embodiments, R³ is the trifluoromethyl group);

(d) B is a methylene or substituted methylene group, wherein a substituent group of B is independently C₁-C₃ alkyl, hydroxy, halogen, amino, or oxo;

(e) D is —NH—, —NR′—, —OC(O)—, —C(O)NH—, —C(O)N(R′)—, —C(O)—, or —S— group, wherein R′ comprises an unsubstituted or substituted C₁-C₁₅ (alternatively, C₁-C₁₀, or C₁-C₅, or C₁-C₃) linear or branched alkyl group;

(f) E is the hydroxy group; and

(g) Q is an aryl or heteroaryl group, each optionally independently substituted with one to three substituent groups, which are independently selected from the group consisting of C₁-C₅ alkyl, C₂-C₅ alkenyl, C₂-C₅ alkynyl, C₁-C₃ alkanoyl, C₃-C₈ cycloalkyl, heterocyclyl, aryl, heteroaryl, C₁-C₅ alkoxy, C₂-C₅ alkenyloxy, C₂-C₅ alkynyloxy, aryloxy, acyl, C₁-C₅ alkoxycarbonyl, aroyl, aminocarbonyl, alkylaminocarbonyl, dialkylaminocarbonyl, aminocarbonyloxy, C₁-C₅ alkylaminocarbonyloxy, C₁-C₅ dialkylaminocarbonyloxy, C₁-C₅ alkanoylamino, C₁-C₅ alkoxycarbonylamino, C₁-C₅ alkylsulfonylamino, aminosulfonyl, C₁-C₅ alkylaminosulfonyl, C₁-C₅ dialkylaminosulfonyl, halogen, hydroxy, carboxy, cyano, trifluoromethyl, trifluoromethoxy, nitro, amino wherein the nitrogen atom is optionally independently mono- or di-substituted by C₁-C₅ alkyl or aryl, ureido wherein either nitrogen atom is optionally independently substituted with C₁-C₅ alkyl, C₁-C₅ alkylthio wherein the sulfur atom is optionally oxidized to a sulfoxide or sulfone, wherein each substituent group of Q is optionally independently substituted with one to three substituent groups selected from the group consisting of C₁-C₃ alkyl, C₁-C₃ alkoxy, acyl, C₁-C₃ silanyloxy, C₁-C₅ alkoxycarbonyl, carboxy, halogen, hydroxy, oxo, cyano, heteroaryl, heterocyclyl, amino wherein the nitrogen atom is optionally independently mono- or di-substituted by C₁-C₅ alkyl or aryl, ureido wherein either nitrogen atom is optionally independently substituted with C₁-C₅ alkyl, and trifluoromethyl.

Non-limiting examples of these compounds include 2-(3,5-difluorobenzylamino)-1,1,1-trifluoro-4-(5-fluoro-2-methoxyphenyl)-4-methylpentan-2-ol; 2-biphenyl-4-ylmethyl-2-hydroxy-1,1,1-trifluoro-4-(5-fluoro-2-methoxyphenyl)-4-methylpentane; 2-(3,5-dimethylbenzylamino)-1,1,1-trifluoro-4-(5-fluoro-2-methoxyphenyl)-4-methylpentan-2-ol; 2-(3-bromobenzylamino)-1,1,1-trifluoro-4-(5-fluoro-2-methoxyphenyl)-4-methylpentan-2-ol; 2-(3,5-dichlorobenzylamino)-1,1,1-trifluoro-4-(5-fluoro-2-methoxyphenyl)-4-methylpentan-2-ol; 2-(3,5-bis-trifluoromethylbenzylamino)-1,1,1-trifluoro-4-(5-fluoro-2-methoxyphenyl)-4-methylpentan-2-ol; 1,1,1-trifluoro-4-(5-fluoro-2-methoxyphenyl)-2-(3-fluoro-5-trifluoromethylbenzylamino)-4-methylpentan-2-ol; 2-(3-chloro-2-fluoro-5-trifluoromethylbenzylamino)-1,1,1-trifluoro-4-(5-fluoro-2-methoxyphenyl)-4-methylpentan-2-ol; 2-(3,5-dibromobenzylamino)-1,1,1-trifluoro-4-(5-fluoro-2-methoxyphenyl)-4-methylpentan-2-ol; 1,1,1-trifluoro-4-(5-fluoro-2-methoxyphenyl)-2-(2-fluoro-3-trifluoromethylbenzylamino)-4-methylpentan-2-ol; 1,1,1-trifluoro-4-(5-fluoro-2-methoxyphenyl)-2-(2-fluoro-5-trifluoromethylbenzylamino)-4-methylpentan-2-ol.

In still another embodiment, the present invention provide a method for producing a stereoisomeric DIGRA compound having Formula Ia or Ib, wherein

(a) A is an aryl, heteroaryl, or C₅-C₁₅ cycloalkyl group, each optionally independently substituted with one to three substituent groups, which are independently selected from the group consisting of C₁-C₅ alkyl, C₂-C₅ alkenyl, C₂-C₅ alkynyl, C₁-C₃ alkanoyl, C₃-C₈ cycloalkyl, heterocyclyl, aryl, heteroaryl, C₁-C₅ alkoxy, C₂-C₅ alkenyloxy, C₂-C₅ alkynyloxy, aryloxy, acyl, C₁-C₅ alkoxycarbonyl, aroyl, aminocarbonyl, alkylaminocarbonyl, dialkylaminocarbonyl, aminocarbonyloxy, C₁-C₅ alkylaminocarbonyloxy, C₁-C₅ dialkylaminocarbonyloxy, C₁-C₅ alkanoylamino, C₁-C₅ alkoxycarbonylamino, C₁-C₅ alkylsulfonylamino, aminosulfonyl, C₁-C₅ alkylaminosulfonyl, C₁-C₅ dialkylaminosulfonyl, halogen, hydroxy, carboxy, cyano, trifluoromethyl, trifluoromethoxy, nitro, amino wherein the nitrogen atom is optionally independently mono- or di-substituted by C₁-C₅ alkyl or aryl, ureido wherein either nitrogen atom is optionally independently substituted with C₁-C₅ alkyl, C₁-C₅ alkylthio wherein the sulfur atom is optionally oxidized to a sulfoxide or sulfone;

(b) R¹ and R² are each independently hydrogen, C₁-C₅ alkyl, C₅-C₁₅ arylalkyl, or R¹ and R² together with the carbon atom they are commonly attached to form a C₃-C₈ spiro cycloalkyl ring;

(c) R³ is the trifluoromethyl group;

(d) B is methylene or substituted methylene group, wherein one or two substituents on the methylene group are independently C₁-C₅ alkyl (or alternatively, C₁-C₃ alkyl), hydroxy, amino, halogen, or oxo group;

(e) D is —NH—, —NR′—, —OC(O), —C(O)NH—, —C(O)N(R′)—, —C(O)—, or —S— group, wherein R′ comprises an unsubstituted or substituted C₁-C₁₅ (alternatively, C₁-C₁₀, or C₁-C₅, or C₁-C₃) linear or branched alkyl group;

(f) E is a hydroxy group; and

(g) Q comprises a quinoline, isoquinoline, pyrrolidine, morpholine, thiomorpholine, piperazine, piperidine, 1H-pyridin-4-one, 1H-pyridin-2-one, 1H-pyridin-4-ylideneamine, 1H-quinolin-4-ylideneamine, pyran, tetrahydropyran, 1,4-diazepane, 2,5-diazabicyclo[2.2.1]heptane, 2,3,4,5-tetrahydrobenzo[b][1,4]diazepine, dihydroquinoline, tetrahydroquinoline, 5,6,7,8-tetrahydro-1H-quinolin-4-one, tetrahydroisoquinoline, decahydroisoquinoline, 2,3-dihydro-1H-isoindole, 2,3-dihydro-1H-indole, chroman, 1,2,3,4-tetrahydroquinoxaline, 1,2-dihydroindazole-3-one, 3,4-dihydro-2H-benzo[1,4]oxazine, 4H-benzo[1,4]thiazine, 3,4-dihydro-2H-benzo[1,4]thiazine, 1,2-dihydrobenzo[d][1,3]oxazin4-one, 3,4-dihydrobenzo[1,4]oxazin4-one, 3H-quinazolin4-one, 3,4-dihydro-1H-quinoxalin-2-one, 1H-quinnolin-4-one, 1H-quinazolin4-one, 1H-[1,5]naphthyridin-4-one, 5,6,7,8-tetrahydro-1H-[1,5]naphthyridin-4-one, 2,3-dihydro-1H-[1,5]naphthyridin-4-one, 1,2-dihydropyrido[3,2-d][1,3]oxazin-4-one, pyrrolo[3,4-c]pyridine-1,3-dione, 1,2-dihydropyrrolo[3,4-c]pyridin-3-one, or tetrahydro[b][1,4]diazepinone group, each optionally independently substituted with one to three substituent groups, wherein each substituent group of Q is independently C₁-C₅ alkyl, C₂-C₅ alkenyl, C₂-C₅ alkynyl, C₃-C₈ cycloalkyl, heterocyclyl, aryl, heteroaryl, C₁-C₅ alkoxy, C₂-C₅ alkenyloxy, C₂-C₅ alkynyloxy, aryloxy, acyl, C₁-C₅ alkoxycarbonyl, C₁-C₅ alkanoyloxy, aminocarbonyl, alkylaminocarbonyl, dialkylaminocarbonyl, aminocarbonyloxy, C₁-C₅ alkylaminocarbonyloxy, C₁-C₅ dialkylaminocarbonyloxy, C₁-C₅ alkanoylamino, C₁-C₅ alkoxycarbonylamino, C₁-C₅ alkylsulfonylamino, C₁-C₅ alkylaminosulfonyl, C₁-C₅ dialkylaminosulfonyl, halogen, hydroxy, carboxy, oxo, cyano, trifluoromethyl, trifluoromethoxy, trifluoromethylthio, nitro, amino wherein the nitrogen atom is optionally independently mono- or di-substituted by C₁-C₅ alkyl, ureido wherein either nitrogen atom is optionally independently substituted with C₁-C₅ alkyl, or C₁-C₅ alkylthio wherein the sulfur atom is optionally oxidized to a sulfoxide or sulfone, wherein each substituent group of Q is optionally independently substituted with one to three substituent groups selected from C₁-C₃ alkyl, C₁-C₃ alkoxy, C₁-C₃ alkoxycarbonyl, acyl, aryl, benzyl, heteroaryl, heterocyclyl, halogen, hydroxy, oxo, cyano, amino wherein the nitrogen atom is optionally independently mono- or di-substituted by C₁-C₅ alkyl, or ureido wherein either nitrogen atom is optionally independently substituted with C₁-C₅ alkyl.

Non-limiting examples of these compounds include 2-((2,6-dimethylmorpholin-4-yl)methylamino)methyl)-1,1,1-trifluoro-4-(5-fluoro-2-methoxyphenyl)-4-methylpentan-2-ol; 6-[(4-(5-fluoro-2-methoxyphenyl)-2-hydroxy-4-methyl-2-trifluoromethylpentyl)amino]-(1H-quinolin-4-one); 3-[(4-(5-fluoro-2-methoxyphenyl)-2-hydroxy-4-methyl-2-trifluoromethylpentyl)amino]-(5-methylpiperidin-4-one); 6-[(4-(5-fluoro-2-methoxyphenyl)-2-hydroxy-4-methyl-2-trifluoromethylpentyl)amino]-(3-methyl-1H-quinolin-4-one); 6-[(4-(5-fluoro-2-methoxyphenyl)-2-hydroxy-4-methyl-2-trifluoromethylpentyl)amino]-(2,3-dihydro-1H-quinolin-4-one); 6-[(4-(4-fluorophenyl)-2-hydroxy-4-methyl-2-trifluoromethylpentyl)amino]-(1H-quinolin-4-one); 5-[(4-(3-fluorophenyl)-2-hydroxy-4-methyl-2-trifluoromethylpentyl)amino]-(1H-quinolin-3-one); 6-[(4-(4-fluoro-2-hydroxyphenyl)-2-hydroxy-4-methyl-2-trifluoromethylpentyl)amino]-(1H-quinolin-4-one); 5-[(4-phenyl-2-hydroxy-4-methyl-2-trifluoromethylpentyl)amino]-(1H-quinolin-3-one); 7-[(4-(5-fluoro-2,3-dihydrobenzofuran-7-yl)-2-hydroxy-4-methyl-2-trifluoromethylpentyl)amino]-(1H-quinolin-4-one); 6-[(4-(5-bromo-2,3-dihydrobenzofuran-7-yl)-2-hydroxy-4-methyl-2-trifluoromethylpentyl)amino]-(1H-quinolin-4-one); 6-[(4-(5-methyl-2,3-dihydrobenzofuran-7-yl)-2-hydroxy-4-methyl-2-trifluoromethylpentyl)amino]-(1H-quinolin-4-one); 6-[(4-(5-chloro-2,3-dihydrobenzofuran-7-yl)-2-hydroxy-4-methyl-2-trifluoromethylpentyl)amino]-(1H-quinolin-4-one); 6-[(4-(2,3-dihydrobenzofuran-7-yl)-2-hydroxy-4-methyl-2-trifluoromethylpentyl)amino]-(1H-quinolin-4-one); 6-[(4-(5-fluoro-2-hydroxyphenyl)-2-hydroxy-4-methyl-2-trifluoromethylpentyl)amino]-(1H-[1,5]naphthyridin-4-one); 1-[(4-(5-fluoro-2-methoxyphenyl)-2-hydroxy-2,4-dimethylpentyl)amino]-(3,5-dimethyl-1H-pyridin-4-one); 6-[(2-hydroxy-4-(2-methoxy-5-thiophen-2-ylphenyl)-4-methyl-2-trifluoromethylpentyl)amino]-(1H-quinolin-4-one); 6-[(4-(6-bromobenzo[1,3]dioxol-4-yl)-2-hydroxy-4-methyl-2-trifluoromethylpentyl)amino]-(1H-quinolin-4-one); 7-[(4-(5-fluoro-2-hydroxyphenyl)-2-hydroxy-4-methyl-2-trifluoromethylpentyl)amino]-(3-methyl-1H-quinolin-4-one); 6-[(2-hydroxy-4-(4-hydroxybiphenyl-3-yl)-4-methyl-2-trifluoromethylpentyl)amino]-(1H-quinolin-4-one); 6-{(4-[5-(3,5-dimethylisoxazol-4-yl)-2-hydroxyphenyl]-2-hydroxy-4-methyl-2-trifluoromethylpentyl)amino}-(1H-quinolin-4-one); 2-[(2-hydroxy-4-(2-hydroxy-5-thiophen-3-ylphenyl)-4-methyl-2-trifluoromethylpentyl)amino]-(1H-quinolin-4-one); 6-{(4-[5-(3,5-dimethylisoxazol-4-yl)-2-methoxyphenyl]-2-hydroxy-4-methyl-2-trifluoromethylpentyl)amino}-(1H-quinolin-4-one); 2-[(2-hydroxy-4-methyl-4-(3-pyridin-3-ylphenyl)-2-trifluoromethylpentyl)amino]-(1H-quinolin-4-one); 6-[(2-hydroxy-4-(2-methoxy-5-thiophen-3-ylphenyl)-4-methyl-2-trifluoromethylpentyl)amino]-(1H-quinolin-4-one); 7-[(4-(5-furan-3-yl-2-methoxyphenyl)-2-hydroxy-4-methyl-2-trifluoromethylpentyl)amino]-(1H-quinolin-4-one); 6-[(2-hydroxy-4-(4-methoxybiphenyl-3-yl)-4-methyl-2-trifluoromethylpentyl)amino]-(1H-quinolin-4-one); 6-[(4-(5-acetyl-2-hydroxyphenyl)-2-hydroxy-4-methyl-2-trifluoromethylpentyl)amino]-(1H-quinolin-4-one); 5-[(3,3,3-trifluoro-2-(6-fluoro-4-methylchroman-4-ylmethyl)-2-hydroxypropyl)amino]-(1H-quinolin-3-one); 5-[(4-{3-[1-(benzyloxyimino)ethyl]phenyl}-2-hydroxy-4-methyl-2-trifluoromethylpentyl)amino]-(1H-quinolin-3-one); 6-[(4-(5-acetyl-2-methoxyphenyl)-2-hydroxy-4-methyl-2-trifluoromethylpentyl)amino]-(1H-quinolin-4-one); 7-[(2-hydroxy-4-{3-[1-(methoxyimino)ethyl]phenyl}-4-methyl-2-trifluoromethylpentyl)amino]-(1H-quinolin-4-one); 6-[(4-(5-bromo-2-hydroxyphenyl)-2-hydroxy-4-methyl-2-trifluoromethylpentyl)amino]-(1H-quinolin-4-one); 6-[(2-hydroxy-4-{3-[1-(hydroxyimino)ethyl]phenyl}-4-methyl-2-trifluoromethylpentyl)amino]-(1H-quinolin-4-one); 6-[(4-(5-bromo-2-methoxyphenyl)-2-hydroxy-4-methyl-2-trifluoromethylpentyl)amino]-(1H-quinolin-4-one); 7-[(4-(3,5-difluorophenyl)-2-hydroxy-4-methyl-2-trifluoromethylpentyl)amino]-(1H-quinolin-4-one); 6-[(4-(3,5-dimethylphenyl)-2-hydroxy-4-methyl-2-trifluoromethylpentyl)amino]-(1H-quinolin-4-one); 6-[{2-hydroxy-4-methyl-4-[3-(2-methyl-[1,3]dioxolan-2-yl)phenyl]-2-trifluoromethylpentyl}amino]-(1H-quinolin-4-one); 7-[(4-(2,3-dihydrobenzofuran-7-yl)-2-hydroxy-4-methyl-2-trifluoromethylpentyl)amino]-(1H-[1,5]naphthyridin-4-one); 6-[(4-(3-[1,3]dioxan-2-ylphenyl)-2-hydroxy-4-methyl-2-trifluoromethylpentyl)amino]-(1H-quinolin-4-one); 6-[{4-[3-(3,5-dimethylisoxazol-4-yl)phenyl]-2-hydroxy-4-methyl-2-trifluoromethylpentyl}amino]-(1H-quinolin-4-one); 1-[(4-(2,3-dihydrobenzofuran-7-yl)-2-hydroxy-4-methyl-2-trifluoromethylpentyl)amino]-(3,5-dimethyl-1H-pyridin-4-one); 1-[(4-(5-fluoro-2-methoxyphenyl)-2-hydroxy-4-methyl-2-trifluoromethylpentyl)amino]-(2-hydroxymethyl-3,5-dimethyl-1H-pyridin-4-one); 6-[(4-hydroxymethyl-1H-quinolin-4-one); 6-[(4-(3-bromophenyl)-2-hydroxy-4-methyl-2-trifluoromethylpentyl)amino]-(1H-quinolin-4-one); 6-[(4-(5-fluoro-2-methoxyphenyl)-2-hydroxy-4-methyl-2-trifluoromethylpentyl]-6-methyl-(1H-quinolin-4-one); 6-[(2-hydroxy-4-(2-hydroxy-5-pyridin-3-ylphenyl)-4-methyl-2-trifluoromethylpentyl)amino]-(1H-quinolin-4-one); and 7-[(2-hydroxy-4-(2-hydroxy-5-pyridin-5-ylphenyl)-4-methyl-2-trifluoromethylpentyl)amino]-(1H-quinolin-4-one).

In still another embodiment, said DIGRA compound has Formula Ia or Ib, wherein A, R¹, R², B, D, E, and Q have the meanings disclosed immediately above, and R³ is hydrogen, C₁-C₈ alkyl, C₂-C₈ alkenyl, C₂-C₈ alkynyl, carbocycle, heterocyclyl, aryl, heteroaryl, carbocycle-C₁-C₈ alkyl, carboxy, alkoxycarbonyl, aryl-C₁-C₈ alkyl, aryl-C₁-C₈ haloalkyl, heterocyclyl-C₁-C₈ alkyl, heteroaryl-C₁-C₈ alkyl, carbocycle-C₂-C₈ alkenyl, aryl-C₂-C₈ alkenyl, heterocyclyl-C₂-C₈ alkenyl, or heteroaryl-C₂-C₈ alkenyl, each optionally independently substituted with one to three substituent groups, wherein each substituent group of R³ is independently C₁-C₅ alkyl, C₂-C₅ alkenyl, C₂-C₅ alkynyl, C₃-C₈ cycloalkyl, phenyl, C₁-C₅ alkoxy, phenoxy, C₁-C₅ alkanoyl, aroyl, C₁-C₅ alkoxycarbonyl, C₁-C₅ alkanoyloxy, aminocarbonyloxy, C₁-C₅ alkylaminocarbonyloxy, C₁-C₅ dialkylaminocarbonyloxy, aminocarbonyl, C₁-C₅ alkylaminocarbonyl, C₁-C₅ dialkylaminocarbonyl, C₁-C₅ alkanoylamino, C₁-C₅ alkoxycarbonylamino, C₁-C₅ alkylsulfonylamino, C₁-C₅ alkylaminosulfonyl, C₁-C₅ dialkylaminosulfonyl, halogen, hydroxy, carboxy, cyano, oxo, trifluoromethyl, nitro, amino wherein the nitrogen atom is optionally independently mono- or di-substituted by C₁-C₅ alkyl, ureido wherein either nitrogen atom is optionally independently substituted with C₁-C₅ alkyl, C₁-C₅ alkylthio wherein the sulfur atom is optionally oxidized to a sulfoxide or sulfone, wherein R³ cannot be trifluoromethyl.

In still another embodiment, the present invention provide a method for producing a stereoisomeric DIGRA compound having Formula Ia or Ib, wherein

(a) A is an aryl, heteroaryl, or C₅-C₁₅ cycloalkyl group, each optionally independently substituted with one to three substituent groups, which are independently selected from the group consisting of C₁-C₅ alkyl, C₂-C₅ alkenyl, C₂-C₅ alkynyl, C₁-C₃ alkanoyl, C₃-C₈ cycloalkyl, heterocyclyl, aryl, heteroaryl, C₁-C₅ alkoxy, C₂-C₅ alkenyloxy, C₂-C₅ alkynyloxy, aryloxy, acyl, C₁-C₅ alkoxycarbonyl, aroyl, aminocarbonyl, alkylaminocarbonyl, dialkylaminocarbonyl, aminocarbonyloxy, C₁-C₅ alkylaminocarbonyloxy, C₁-C₅ dialkylaminocarbonyloxy, C₁-C₅ alkanoylamino, C₁-C₅ alkoxycarbonylamino, C₁-C₅ alkylsulfonylamino, aminosulfonyl, C₁-C₅ alkylaminosulfonyl, C₁-C₅ dialkylaminosulfonyl, halogen, hydroxy, carboxy, cyano, trifluoromethyl, trifluoromethoxy, nitro, amino wherein the nitrogen atom is optionally independently mono- or di-substituted by C₁-C₅ alkyl or aryl, ureido wherein either nitrogen atom is optionally independently substituted with C₁-C₅ alkyl, C₁-C₅ alkylthio wherein the sulfur atom is optionally oxidized to a sulfoxide or sulfone;

(b) R¹ and R² are each independently hydrogen or C₁-C₅ alkyl, or R¹ and R² together with the carbon atom they are commonly attached to form a C₃-C₈ spiro cycloalkyl ring;

(c) R³ is the trifluoromethyl group;

(d) B is a methylene group;

(e) D is —NH—, —NR′—, —OC(O)—, —C(O)NH—, —C(O)N(R′)—, —C(O)—, or —S—, wherein R′ comprises an unsubstituted or substituted C₁-C₁₅ (alternatively, C₁-C₁₀, or C₁-C₅, or C₁-C₃) linear or branched alkyl group;

(f) E is the hydroxy group; and

(g) Q comprises an optionally substituted phenyl group having the formula

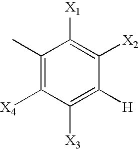

wherein X₁, X₂, X₃ and X₄ are each independently selected from the group consisting of hydrogen, halogen, hydroxy, trifluoromethyl, trifluoromethoxy, C₁-C₅ alkyl, C₂-C₅ alkenyl, C₂-C₅ alkynyl, C₁-C₅ alkoxy, C₁-C₅ alkylthio wherein the sulfur atom is optionally oxidized to a sulfoxide or sulfone, C₁-C₅ alkanoyl, C₁-C₅ alkoxycarbonyl, C₁-C₅ acyloxy, C₁-C₅ alkanoylamino, C₁-C₅ carbamoyloxy, urea, aryl, and amino wherein the nitrogen atom may be independently mono- or di-substituted by C₁-C₅ alkyl, and wherein said aryl group is optionally substituted by one or more hydroxy or C₁-C₅ alkoxy groups, and wherein either nitrogen atom of the urea group may be independently substituted by C₁-C₅ alkyl; or Q is an aromatic 5- to 7-membered monocyclic ring having from one to four heteroatoms in the ring independently selected from nitrogen, oxygen, and sulfur, optionally independently substituted with one to three substituent groups selected from the group consisting of hydrogen, halogen, hydroxy, trifluoromethyl, trifluoromethoxy, C₁-C₅ alkyl, C₂-C₅ alkenyl, C₂-C₅ alkynyl, C₁-C₅ alkoxy, C₁-C₅ alkylthio wherein the sulfur atom is optionally oxidized to a sulfoxide or sulfone, C₁-C₅ alkanoyl, C₁-C₅ alkoxycarbonyl, C₁-C₅ acyloxy, C₁-C₅ alkanoylamino, C₁-C₅ carbamoyloxy, urea, aryl optionally substituted by one or more hydroxy or C₁-C₅ alkoxy groups, and amino wherein the nitrogen atom may be independently mono- or di-substituted by C₁-C₅ alkyl, and wherein either nitrogen atom of the urea group may be independently substituted by C₁-C₅ alkyl.

Non-limiting examples of these compounds include 1-[4-(5-fluoro-2-hydroxyphenyl)-2-hydroxy-4-methyl-2-trifluoromethyl-pentylamino]-(3,5-dichlorobenzene); 1-[4-(5-fluoro-2-hydroxyphenyl)-2-hydroxy-4-methyl-2-trifluoromethyl-pentylamino]-(3-chlorobenzene); 5-(5-fluoro-2-hydroxyphenyl)-3-hydroxy-5-methyl-3-trifluoromethyl-hexanoic acid-(2-chlorophenyl)amide; 5-(5-fluoro-2-hydroxyphenyl)-3-hydroxy-5-methyl-3-trifluoromethyl-hexanoic acid-(2,6-dichloropyrimidin-4-yl)amide; 5-(5-fluoro-2-hydroxyphenyl)-3-hydroxy-5-methyl-3-trifluoromethyl-hexanoic acid-(2,6-dichloropyridin-4-yl)amide; 5-(5-fluoro-2-hydroxyphenyl)-3-hydroxy-5-methyl-3-trifluoromethyl-hexanoic acid-(2,3-dichlorophenyl)amide; 5-(5-fluoro-2-hydroxyphenyl)-2-hydroxy-4-methyl-2-trifluoromethyl-hexanoic acid-(3,5-dimethylphenyl)amide; 5-(5-fluoro-2-hydroxyphenyl)-3-hydroxy-5-methyl-3-trifluoromethyl-hexanoic acid-(3,5-bis-trifluoromethylphenyl)amide; 5-(5-fluoro-2-hydroxyphenyl)-3-hydroxy-5-methyl-3-trifluoromethyl-hexanoic acid-(2,5-dichlorophenyl)amide; 5-(5-fluoro-2-hydroxyphenyl)-3-hydroxy-5-methyl-3-trifluoromethyl-hexanoic acid-(3-bromophenyl)amide; 5-(5-fluoro-2-hydroxyphenyl)-3-hydroxy-5-methyl-3-trifluoromethyl-hexanoic acid-(3,5-difluorophenyl)amide; 5-(5-fluoro-2-hydroxyphenyl)-3-hydroxy-5-methyl-3-trifluoromethyl-hexanoic acid-(3,5-dibromophenyl)-amide.

In still another embodiment, the present invention provides a method for producing a DIGRA compound having Formula Ia or Ib, wherein:

(a) A is an aryl or heteroaryl group, each optionally independently substituted with one to three substituent groups, which are independently selected from the group consisting of C₁-C₅ alkyl, C₂-C₅ alkenyl, C₂-C₅ alkynyl, C₁-C₃ alkanoyl, C₃-C₈ cycloalkyl, heterocyclyl, aryl, heteroaryl, C₁-C₅ alkoxy, C₂-C₅ alkenyloxy, C₂-C₅ alkynyloxy, aryloxy, acyl, C₁-C₅ alkoxycarbonyl, aroyl, aminocarbonyl, alkylaminocarbonyl, dialkylaminocarbonyl, aminocarbonyloxy, C₁-C₅ alkylaminocarbonyloxy, C₁-C₅ dialkylaminocarbonyloxy, C₁-C₅ alkanoylamino, C₁-C₅ alkoxycarbonylamino, C₁-C₅ alkylsulfonylamino, aminosulfonyl, C₁-C₅ alkylaminosulfonyl, C₁-C₅ dialkylaminosulfonyl, halogen, hydroxy, carboxy, cyano, trifluoromethyl, trifluoromethoxy, nitro, amino wherein the nitrogen atom is optionally independently mono- or di-substituted by C₁-C₅ alkyl or aryl, ureido wherein either nitrogen atom is optionally independently substituted with C₁-C₅ alkyl, C₁-C₅ alkylthio wherein the sulfur atom is optionally oxidized to a sulfoxide or sulfone;

(b) R¹ and R² are each independently hydrogen or C₁-C₅ alkyl;

(c) R³ is C₁-C₈ alkyl, C₂-C₈ alkenyl, C₂-C₈ alkynyl, carbocycle, heterocyclyl, aryl, heteroaryl, carbocycle-C₁-C₈ alkyl, aryl-C₁-C₈ alkyl, aryl-C₁-C₈ haloalkyl, heterocyclyl-C₁-C₈ alkyl, heteroaryl-C₁-C₈ alkyl, carbocycle-C₂-C₈ alkenyl, aryl-C₂-C₈ alkenyl, heterocyclyl-C₂-C₈ alkenyl, or heteroaryl-C₂-C₈ alkenyl, each optionally independently substituted with one to three substituent groups, wherein each substituent group of R³ is independently C₁-C₅ alkyl, C₂-C₅ alkenyl, C₂-C₅ alkynyl, C₃-C₈ cycloalkyl, phenyl, C₁-C₅ alkoxy, phenoxy, C₁-C₅ alkanoyl, aroyl, C₁-C₅ alkoxycarbonyl, C₁-C₅ alkanoyloxy, aminocarbonyloxy, C₁-C₅ alkylaminocarbonyloxy, C₁-C₅ dialkylaminocarbonyloxy, aminocarbonyl, C₁-C₅ alkylaminocarbonyl, C₁-C₅ dialkylaminocarbonyl, C₁-C₅ alkanoylamino, C₁-C₅ alkoxycarbonylamino, C₁-C₅ alkylsulfonylamino, C₁-C₅ alkylaminosulfonyl, C₁-C₅ dialkylaminosulfonyl, halogen, hydroxy, carboxy, cyano, oxo, trifluoromethyl, nitro, amino wherein the nitrogen atom is optionally independently mono- or di-substituted by C₁-C₅ alkyl, ureido wherein either nitrogen atom is optionally independently substituted with C₁-C₅ alkyl, or C₁-C₅ alkylthio wherein the sulfur atom is optionally oxidized to a sulfoxide or sulfone, wherein R³ cannot be trifluoromethyl;

(d) B is a methylene or substituted methylene group, wherein one or two substituent groups of B is independently C₁-C₅ alkyl (or alternatively, C₁-C₃ alkyl), hydroxy, halogen, amino, or oxo;

(e) D is —NH— or —NR′—, wherein R′ comprises an unsubstituted or substituted C₁-C₁₅ (alternatively, C₁-C₁₀, or C₁-C₅, or C₁-C₃) linear or branched alkyl group;

(f) E is the hydroxy group; and

(g) Q comprises an azaindolyl group optionally independently substituted with one to three substituent groups, wherein each substituent group of Q is independently C₁-C₅ alkyl, C₂-C₅ alkenyl, C₂-C₅ alkynyl, C₃-C₈ cycloalkyl, heterocyclyl, aryl, heteroaryl, C₁-C₅ alkoxy, C₂-C₅ alkenyloxy, C₂-C₅ alkynyloxy, aryloxy, acyl, C₁-C₅ alkoxycarbonyl, C₁-C₅ alkanoyloxy, aminocarbonyl, alkylaminocarbonyl, dialkylaminocarbonyl, aminocarbonyloxy, C₁-C₅ alkylaminocarbonyloxy, C₁-C₅ dialkylaminocarbonyloxy, C₁-C₅ alkanoylamino, C₁-C₅ alkoxycarbonylamino, C₁-C₅ alkylsulfonylamino, aminosulfonyl, C₁-C₅ alkylaminosulfonyl, C₁-C₅ dialkylaminosulfonyl, halogen, hydroxy, carboxy, cyano, trifluoromethyl, trifluoromethoxy, trifluoromethylthio, nitro, amino wherein the nitrogen atom is optionally independently mono- or di-substituted by C₁-C₅ alkyl, ureido wherein either nitrogen atom is optionally independently substituted with C₁-C₅ alkyl, or C₁-C₅ alkylthio wherein the sulfur atom is optionally oxidized to a sulfoxide or sulfone, wherein each substituent group of Q is optionally independently substituted with one to three substituent groups selected from C₁-C₃ alkyl, C₁-C₃ alkoxy, halogen, hydroxy, oxo, cyano, amino, or trifluoromethyl.

Non-limiting examples of these compounds include [1,1,1-trifluoro-4-(5-fluoro-2-methoxyphen-1-yl)-4-methyl-2-(1H-pyrrolo[2,3-c]pyridin-2-ylmethyl)amino]pentan-2-ol; [1,1,1-trifluoro-4-(5-fluoro-2-methoxyphenyl)-4-methyl-2-(1H-pyrrolo[2,3-b]pyridin-2-ylmethyl)amino]pentan-2-ol; [1,1,1-trifluoro-4-(5-fluoro-2-methoxyphenyl)-4-methyl-2-(1H-pyrrolo[3,2-c]pyridin-2-ylmethyl)amino]pentan-2-ol; [1,1,1-trifluoro-4-(5-fluoro-2-methoxyphenyl)-4-methyl-2-(1H-pyrrolo[3,2-b]pyridin-2-ylmethyl)amino]pentan-2-ol; [1,1,1-trifluoro-4-(3-fluorophenyl)-4-methyl-2-(1H-pyrrolo[2,3-c]pyridin-2-ylmethyl)amino]pentan-2-ol; [1,1,1-trifluoro-4-(4-fluorophenyl)-4-methyl-2-(1H-pyrrolo[2,3-c]pyridin-2-ylmethyl)amino]pentan-2-ol; [4-(2,3-dihydrobenzofuran-7-yl)-1,1,1-trifluoro-4-methyl-2-(1H-pyrrolo[2,3-c]pyridin-2-ylmethyl)amino]pentan-2-ol; [4-(2,3-dihydrobenzofuran-7-yl)-1,1,1-trifluoro-4-methyl-2-(1H-pyrrolo[3,2-c]pyridin-2-ylmethyl)amino]pentan-2-ol; [1,1,1-trifluoro-4-methyl-4-phenyl-2-(1H-pyrrolo[2,3-c]pyridine-2-ylmethyl)amino]pentan-2-ol; [1,1,1-trifluoro-4-(4-fluoro-2-methoxyphenyl)-4-methyl-2-(1H-pyrrolo[2,3-c]pyridin-2-ylmethyl)amino]pentan-2-ol; [1,1,1-trifluoro-4-(4-fluoro-2-methoxyphenyl)-4-methyl-2-(1H-pyrrolo[3,2-c]pyridin-2-ylmethyl)amino]pentan-2-ol; [1,1,1-trifluoro-4-methyl-4-phenyl-2-(1H-pyrrolo[3,2-c]pyridin-2-ylmethyl)amino]pentan-2-ol; [1,1,1-trifluoro-4-(4-fluorophenyl)-4-methyl-2-(1H-pyrrolo[3,2-c]pyridin-2-ylmethyl)amino]pentan-2-ol; [1,1,1-trifluoro-4-(5-fluoro-2-methylphenyl)-4-methyl-2-(1H-pyrrolo[2,3-c]pyridin-2-ylmethyl)amino]pentan-2-ol; [1,1,1-trifluoro-4-(5-fluoro-2-methoxyphenyl)-4-methyl-2-(3-methyl-1H-pyrrolo[2,3-c]pyridin-2-ylmethyl)amino]pentan-2-ol; and [1,1,1-trifluoro-4-(5-fluoro-2,3-dihydrobenzofuran-7-yl)-4-methyl-2-(1H-pyrrolo[2,3-c]pyridine-2-ylmethyl)amino]pentan-2-ol.

In still another embodiment, the present invention provides a method for producing a DIGRA compound having Formula Ia or Ib, wherein

(a) A is cycloalkyl, an aryl, or heteroaryl group, each optionally independently substituted with one to three substituent groups, which are independently selected from the group consisting of C₁-C₅ alkyl, C₂-C₅ alkenyl, C₂-C₅ alkynyl, C₁-C₃ alkanoyl, C₃-C₈ cycloalkyl, heterocyclyl, aryl, heteroaryl, C₁-C₅ alkoxy, C₂-C₅ alkenyloxy, C₂-C₅ alkynyloxy, aryloxy, acyl, C₁-C₅ alkoxycarbonyl, aroyl, aminocarbonyl, alkylaminocarbonyl, dialkylaminocarbonyl, aminocarbonyloxy, C₁-C₅ alkylaminocarbonyloxy, C₁-C₅ dialkylaminocarbonyloxy, C₁-C₅ alkanoylamino, C₁-C₅ alkoxycarbonylamino, C₁-C₅ alkylsulfonylamino, aminosulfonyl, C₁-C₅ alkylaminosulfonyl, C₁-C₅ dialkylaminosulfonyl, halogen, hydroxy, carboxy, cyano, trifluoromethyl, trifluoromethoxy, nitro, amino wherein the nitrogen atom is optionally independently mono- or di-substituted by C₁-C₅ alkyl or aryl, ureido wherein either nitrogen atom is optionally independently substituted with C₁-C₅ alkyl, C₁-C₅ alkylthio wherein the sulfur atom is optionally oxidized to a sulfoxide or sulfone;

(b) R¹ and R² are each independently hydrogen or C₁-C₅ alkyl, or R¹ and R² together with the carbon atom they are commonly attached to form a C₃-C₈ spiro cycloalkyl ring;

(c) R³ is the trifluoromethyl group;

(d) B is a methylene or substituted methylene group having one or two substituents independently selected from the group consisting of C₁-C₃ alkyl, hydroxy, halogen, amino, and oxo;

(e) D is —NH— or —NR′—, wherein R′ comprises an unsubstituted or substituted C₁-C₁₅ (alternatively, C₁-C₁₀, or C₁-C₅, or C₁-C₃) linear or branched alkyl group;

(f) E is the hydroxy group; and

(g) Q comprises a heteroaryl group optionally independently substituted with one to three substituent groups, which are independently selected from the group consisting of C₁-C₅ alkyl, C₂-C₅ alkenyl, C₂-C₅ alkynyl, C₁-C₃ alkanoyl, C₃-C₈ cycloalkyl, heterocyclyl, aryl, heteroaryl, C₁-C₅ alkoxy, C₂-C₅ alkenyloxy, C₂-C₅ alkynyloxy, aryloxy, acyl, C₁-C₅ alkoxycarbonyl, aroyl, aminocarbonyl, alkylaminocarbonyl, dialkylaminocarbonyl, aminocarbonyloxy, C₁-C₅ alkylaminocarbonyloxy, C₁-C₅ dialkylaminocarbonyloxy, C₁-C₅ alkanoylamino, C₁-C₅ alkoxycarbonylamino, C₁-C₅ alkylsulfonylamino, aminosulfonyl, C₁-C₅ alkylaminosulfonyl, C₁-C₅ dialkylaminosulfonyl, halogen, hydroxy, carboxy, cyano, trifluoromethyl, trifluoromethoxy, nitro, amino wherein the nitrogen atom is optionally independently mono- or di-substituted by C₁-C₅ alkyl or aryl, ureido wherein either nitrogen atom is optionally independently substituted with C₁-C₅ alkyl, C₁-C₅ alkylthio wherein the sulfur atom is optionally oxidized to a sulfoxide or sulfone, wherein each substituent group of Q is optionally independently substituted with one to three substituent groups selected from the group consisting of C₁-C₃ alkyl, C₁-C₃ alkoxy, acyl, C₁-C₃ silanyloxy, C₁-C₅ alkoxycarbonyl, carboxy, halogen, hydroxy, oxo, cyano, heteroaryl, heterocyclyl, amino wherein the nitrogen atom is optionally independently mono- or di-substituted by C₁-C₅ alkyl or aryl, ureido wherein either nitrogen atom is optionally independently substituted with C₁-C₅ alkyl, or trifluoromethyl.

Non-limiting examples of these compounds include 4-cyclohexyl-1,1,1-trifluoro-4-methyl-2-[(2-methyl-quinolin-4-yl)amino]pentan-2-ol; 1,1,1-trifluoro-4-(5-fluoro-2-methoxyphen-1-yl)-4-methyl-2-[(3-methyl-1H-pyrrolo[3,2-c]pyridin-2-ylmethyl)amino]pentan-2-ol; 1,1,1-trifluoro-4-(5-fluoro-2,3-dihydrobenzofuran-7-yl)-4-methyl-2-[(1H-pyrrolo[3,2-c]pyridin-2-ylmethyl)amino]pentan-2-ol; 1,1,1-trifluoro-4-(5-fluoro-2-methylphen-1-yl)-4-methyl-2-[(3-methyl-1H-pyrrolo[2,3-c]pyridin-2-ylmethyl)amino]pentan-2-ol; 2-[(4,6-dimethyl-1H-pyrrolo[3,2-c]pyridin-2-ylmethyl)amino]-1,1,1-trifluoro-4-(5-fluoro-2-methoxyphenyl)-4-methylpentan-2-ol; 2-[(5,7-dimethyl-1H-pyrrolo[2,3-c]pyridin-2-ylmethyl)amino]-1,1,1-trifluoro-4-(5-fluoro-2-methoxyphenyl)-4-methylpentan-2-ol; 1,1,1-trifluoro-4-(5-fluoro-2-methoxyphenyl)-4-methyl-2-[(6-methyl-1H-pyrrolo[3,2-c]pyridin-2-ylmethyl)amino]pentan-2-ol; 1,1,1-trifluoro-4-(5-fluoro-2-methoxyphenyl)-4-methyl-2-[(4-methyl-1H-pyrrolo[3,2-c]pyridin-2-ylmethyl)amino]pentan-2-ol; 1,1,1-trifluoro-4-(5-fluoro-2-methoxyphenyl)-4-methyl-2-[(5H-pyrrolo[3,2-d]pyrimidin-6-ylmethyl)amino]pentan-2-ol; 1,1,1-trifluoro-4-(5-fluoro-2-methoxyphenyl)-4-methyl-2-[(thieno[2,3-d]pyridazin-2-ylmethyl)amino]pentan-2-ol; 1,1,1-trifluoro-4-(5-fluoro-2-methoxyphenyl)-4-methyl-2-[(5H-pyrrolo[3,2-c]pyridazin-6-ylmethyl)amino]pentan-2-ol; 1,1,1-trifluoro-4-(5-fluoro-2-methoxyphenyl)-4-methyl-2-[(2-methyl-5H-pyrrolo[3,2-d]pyrimidin-6-ylmethyl)amino]pentan-2-ol; 1,1,1-trifluoro-4-(5-fluoro-2-methylphenyl)-4-methyl-2-[(1H-pyrrolo[2,3-d]pyridazin-2-ylmethyl)amino]pentan-2-ol; 2-[(4,6-dimethyl-H-pyrrolo[3,2-c]pyridin-2-ylmethyl)amino]-1,1,1-trifluoro-4-(5-fluoro-2-methylphenyl)-4-methylpentan-2-ol; 4-(5-chloro-2,3-dihydrobenzofuran-7-yl)-2-[(4,6-dimethyl-1H-pyrrolo[3,2-c]pyridin-2-ylmethyl)amino]-1,1,1-trifluoro-4-methylpentan-2-ol; 4-(5-chloro-2,3-dihydrobenzofuran-7-yl)-1,1,1-trifluoro-4-methyl-2-[(3-methyl-1H-pyrrolo[2,3-c]pyridin-2-ylmethyl)amino]pentan-2-ol; 1,1,1-trifluoro-4-(5-fluoro-2-methylphenyl)-4-methyl-2-[(5H-pyrrolo[3,2-c]-pyridazin-6-ylmethyl)amino]pentan-2-ol; 4-(5-chloro-2,3-dihydrobenzofuran-7-yl)-1,1,1-trifluoro-4-methyl-2-[(5H-pyrrolo[3,2-c]pyridazin-6-ylmethyl)amino]pentan-2-ol; 4-(5-chloro-2,3-dihydrobenzofuran-7-yl)-1,1,1-trifluoro-4-methyl-2-[(1H-pyrrolo[2,3-d]pyridazin-2-ylmethyl)amino]pentan-2-ol; 1,1,1-trifluoro-4-(5-fluoro-2-methoxyphenyl)-2-[(7-fluoro-1H-pyrrolo[2,3-c]pyridin-2-ylmethyl)amino]-4-methylpentan-2-ol; 1,1,1-trifluoro-4-(5-fluoro-2-methoxyphenyl)-4-methyl-2-[(4-methyl-1H-pyrrolo[2,3-c]pyridin-2-ylmethyl)amino]pentan-2-ol; 2-[(5,7-dichloro-1H-pyrrolo[2,3-c]pyridin-2-ylmethyl)amino]-1,1,1-trifluoro-4-(5-fluoro-2-methoxyphenyl)-4-methylpentan-2-ol; 1,1,1-trifluoro-4-(5-fluoro-2-methoxyphenyl)-4-methyl-2-[(5-trifluoromethyl-1H-pyrrolo[2,3-c]pyridin-2-ylmethyl)amino]pentan-2-ol; 1,1,1-trifluoro-4-(5-fluoro-2-methoxyphenyl)-2-[(5-methoxy-1H-pyrrolo[2,3-c]pyridin-2-ylmethyl)amino]-4-methylpentan-2-ol; 1,1,1-trifluoro-4-(5-fluoro-2-methylphenyl)-4-methyl-2-[(4-methyl-1H-pyrrolo[2,3-c]pyridin-2-ylmethyl)amino]pentan-2-ol; 1,1,1-trifluoro-4-(5-fluoro-2-methylphenyl)-2-[(5-isopropoxy-1H-pyrrolo[2,3-c]pyridin-2-ylmethyl)amino]-4-methylpentan-2-ol; 1,1,1-trifluoro-4-(5-fluoro-2-methylphenyl)-2-[(5-methoxy-1H-pyrrolo[2,3-c]pyridin-2-ylmethyl)amino]-4-methylpentan-2-ol; 4-(5-chloro-2,3-dihydrobenzofuran-7-yl)-1,1,1-trifluoro-2-[(5-methoxy-1H-pyrrolo[2,3-c]pyridin-2-ylmethyl)amino]-4-methylpentan-2-ol; 1,1,1-trifluoro-4-(5-fluoro-2-methylphenyl)-2-[(7-fluoro-1H-pyrrolo[2,3-c]pyridin-2-ylmethyl)amino]-4-methylpentan-2-ol; 4-(5-chloro-2,3-dihydrobenzofuran-7-yl)-1-trifluoro-4-methyl-2-[(5-trifluoromethyl-1H-pyrrolo[2,3-c]pyridin-2-ylmethyl)amino]pentan-2-ol; 1,1,1-trifluoro-4-(5-fluoro-2-methylphenyl)-4-methyl-2-[(5-trifluoromethyl-1H-pyrrolo[2,3-c]pyridin-2-ylmethyl)amino]pentan-2-ol; 4-(5-chloro-2,3-dihydrobenzofuran-7-yl)-1,1,1-trifluoro-2-[(5-isopropoxy-1H-pyrrolo[2,3-c]pyridin-2-ylmethyl)amino]-4-methylpentan-2-ol; 4-(5-chloro-2,3-dihydrobenzofuran-7-yl)-1,1,1-trifluoro-2-[(7-fluoro-1H-pyrrolo[2,3-c]pyridin-2-ylmethyl)amino]-4-methylpentan-2-ol; 4-(5-chloro-2,3-dihydrobenzofuran-7-yl)-2-[(5-dimethylamino-1H-pyrrolo[2,3-c]pyridin-2-ylmethyl)amino]-1,1,1-trifluoro-4-methylpentan-2-ol; 4-(5-chloro-2,3-dihydrobenzofuran-7-yl)-1,1,1-trifluoro-4-methyl-2-[(5-piperidin-1-yl-1H-pyrrolo[2,3-c]pyridin-2-ylmethyl)amino]pentan-2-ol; 4-(5-chloro-2,3-dihydrobenzofuran-7-yl)-1,1,1-trifluoro-4-methyl-2-[(5-morpholin-4-yl-1H-pyrrolo[2,3-c]pyridin-2-ylmethyl)amino]pentan-2-ol; 1,1,1-trifluoro-4-(5-fluoro-2-methylphenyl)-4-methyl-2-[(5-piperidin-1-yl-1H-pyrrolo[2,3-c]pyridin-2-ylmethyl)amino]pentan-2-ol; 4-(5-chloro-2,3-dihydrobenzofuran-7-yl)-2-[(5-ethoxy-1H-pyrrolo[2,3-c]pyridin-2-ylmethyl)amino]-1,1,1-trifluoro-4-methylpentan-2-ol; 2-[(5-benzyloxy-1H-pyrrolo[2,3-c]pyridin-2-ylmethyl)amino]-1,1,1-trifluoro-4-(5-fluoro-2-methylphenyl)-4-methylpentan-2-ol; 2-[(5-benzyloxy-1H-pyrrolo[2,3-c]pyridin-2-ylmethyl)amino]-4-(5-chloro-2,3-dihydrobenzofuran-7-yl)-1,1,1-trifluoro-4-methylpentan-2-ol; 1,1,1-trifluoro-4-(5-fluoro-2-methoxyphenyl)-2-[(5-chloro-1H-pyrrolo[2,3-c-]pyridin-2-ylmethyl)amino]-4-methylpentan-2-ol; 1,1,1-trifluoro-4-(5-fluoro-2-methoxyphenyl)-4-methyl-2-[(5-(methylamino)-1H-pyrrolo[2,3-c]pyridin-2-ylmethyl)amino]pentan-2-ol; 1,1,1-trifluoro-4-(5-fluoro-2-methoxyphenyl)-4-methyl-2-[(5-amino-1H-pyrrolo[2,3-c]pyridin-2-ylmethyl)amino]pentan-2-ol; 1,1,1-trifluoro-4-(5-fluoro-2-methylphenyl)-4-methyl-2-[(6-amino-1H-pyrrol-o[2,3-c]pyridin-2-ylmethyl)amino]pentan-2-ol; 4-(5-chloro-2,3-dihydrobenzofuran-7-yl)-1,1,1-trifluoro-2-[(5-amino-1H-pyrrolo[2,3-c]pyridin-2-ylmethyl)amino]-4-methylpentan-2-ol; 4-(5-chloro-2,3-dihydrobenzofuran-7-yl)-1,1,1-trifluoro-4-methyl-2-[(5-methylamino-1H-pyrrolo[2,3-c]pyridin-2-ylmethyl)amino]pentan-2-ol; 4-(5-bromo-2,3-dihydrobenzofuran-7-yl)-1,1,1-trifluoro-4-methyl-2-[(1H-pyrrolo[2,3-c]pyridin-2-ylmethyl)amino]pentan-2-ol; 1,1,1-trifluoro-4-methyl-4-(5-methyl-2,3-dihydrobenzofuran-7-yl)-2-[(1H-pyrrolo[2,3-c]pyridin-2-ylmethyl)amino]pentan-2-ol; 4-(5-chloro-2,3-dihydrobenzofuran-7-yl)-1,1,1-trifluoro-4-methyl-2-[(1H-pyrrolo[2,3-c]pyridin-2-ylmethyl)amino]pentan-2-ol; 1,1,1-trifluoro-4-(5-fluoro-2-methoxyphenyl)-4-methyl-2-[(pyrrolo[2,3-b]pyridin-1-ylmethyl)amino]pentan-2-ol; 1,1,1-trifluoro-4-(5-fluoro-2-methoxyphenyl)-4-methyl-2-[(6-oxy-1H-pyrrolo[2,3-c]pyridin-2-ylmethyl)amino]pentan-2-ol; 1,1,1-trifluoro-4-(5-fluoro-2-methoxyphenyl)-4-methyl-2-[(pyrrolo[2,3-c]pyridin-1-ylmethyl)amino]pentan-2-ol; 2-[(benzo[b]thiophen-2-ylmethyl)amino]-1,1,1-trifluoro-4-(5-fluoro-2-methoxyphenyl)-4-methylpentan-2-ol; 1,1,1-trifluoro-4-(5-fluoro-2-methoxyphenyl)-4-methyl-2-[(thieno[2,3-c]pyridin-2-ylmethyl)amino]pentan-2-ol; 1,1,1-trifluoro-4-(5-fluoro-2-methoxyphenyl)-2-[(indazol-1-ylmethyl)amino]-4-methylpentan-2-ol; 1,1,1-trifluoro-4-(5-fluoro-2-methoxyphenyl)-4-methyl-2-[(pyrazolo[1,5-a]pyridin-2-ylmethyl)amino]pentan-2-ol; 1,1,1-trifluoro-4-(5-fluoro-2-methoxyphenyl)-2-[(furo[2,3-c]pyridin-2-ylmethyl)amino]-4-methylpentan-2-ol; 1,1,1-trifluoro-4-methyl-4-(5-methyl-2,3-dihydrobenzofuran-7-yl)-2-[(1H-pyrrolo[3,2-c]pyridin-2-ylmethyl)amino]pentan-2-ol; 4-(5-chloro-2,3-dihydrobenzofuran-7-yl)-1,1,1-trifluoro-4-methyl-2-[(1H-pyrrolo[3,2-c]pyridin-2-ylmethyl)amino]pentan-2-ol; 4-(5-bromo-2,3-dihydrobenzofuran-7-yl)-1,1,1-trifluoro-4-methyl-2-[(1H-pyrrolo[3,2-c]pyridin-2-ylmethyl)amino]pentan-2-ol; 2-[(3-dimethylaminomethyl-1H-pyrrolo[3,2-c]pyridin-2-ylmethyl)amino]-1,1,1-trifluoro-4-(5-fluoro-2-methoxyphenyl)-4-methylpentan-2-ol; 1,1,1-trifluoro-4-(5-fluoro-2-methoxyphenyl)-2-[(furo[3,2-c]pyridin-2-ylmethyl)amino]-4-methylpentan-2-ol; 4-(5-chloro-2,3-dihydrobenzofuran-7-yl)-1,1,1-trifluoro-4-methyl-2-[(pyrrolo[3,2-b]pyridin-1-ylmethyl)amino]pentan-2-ol; 1,1,1-trifluoro-4-(5-fluoro-2-methoxyphenyl)-4-methyl-2-[(thieno[3,2-c]pyridin-2-ylmethyl)amino]pentan-2-ol; 4-(5-chloro-2,3-dihydrobenzofuran-7-yl)-1,1,1-trifluoro-4-methyl-2-[(thieno[3,2-c]pyridin-2-ylmethyl)amino]pentan-2-ol; 1,1,1-trifluoro-4-(5-fluoro-2-methylphenyl)-4-methyl-2-[(pyrrolo[3,2-b]pyridin-1-ylmethyl)amino]pentan-2-ol; 1,1,1-trifluoro-4-(5-fluoro-2-methylphenyl)-4-methyl-2-[(thieno[3,2-c]pyridin-2-ylmethyl)amino]pentan-2-ol; 2-[4-(5-fluoro-2-hydroxyphenyl)-2-hydroxy-4-methyl-2-trifluoromethylpentylamino]-1H-indole-6-carboxylic acid; 2-[4-(5-fluoro-2-hydroxyphenyl)-2-hydroxy-4-methyl-2-trifluoromethylpentylamino]-1H-indole-6-carboxylic acid dimethylamide; 2-[4-(5-fluoro-2-methoxyphenyl)-2-hydroxy-4-methyl-2-trifluoromethylpentylamino]-1H-indole-6-carboxylic acid dimethylamide; 2-[4-(5-fluoro-2-hydroxyphenyl)-2-hydroxy-4-methyl-2-trifluoromethylpentylamino]-1H-indole-6-carboxylic acid amide; 2-[4-(5-fluoro-2-methoxyphenyl)-2-hydroxy-4-methyl-2-trifluoromethylpentylamino]-1H-indole-6-carboxylic acid amide; 1,1,1-trifluoro-4-(4-fluoro-2-methoxyphenyl)-2-[(7-fluoro-4-methyl-1H-indol-2-ylmethyl)amino]-4-methylpentan-2-ol; 2-[4-(5-fluoro-2-methoxyphenyl)-2-hydroxy-4-methyl-2-trifluoromethylpentylamino]-1H-indole-5-carboxylic acid-2-trimethylsilanylethyl ester; 2-[4-(5-fluoro-2-methoxyphenyl)-2-hydroxy-4-methyl-2-trifluoromethylpentylamino]-1H-indole-5-carboxylic acid; 2-[4-(5-fluoro-2-methoxyphenyl)-2-hydroxy-4-methyl-2-trifluoromethylpentylamino]-1H-indole-5-carboxylic acid methylamide; 2-[4-(5-bromo-2,3-dihydrobenzofuran-7-yl)-2-hydroxy-4-methyl-2-trifluoromethylpentylamino]-1H-indole-5-carboxylic acid; 2-[4-(5-bromo-2,3-dihydrobenzofuran-7-yl)-2-hydroxy-4-methyl-2-trifluoromethylpentylamino]-1H-indole-5-carboxylic acid amide; 2-[4-(5-bromo-2,3-dihydrobenzofuran-7-yl)-2-hydroxy-4-methyl-2-trifluoromethylpentylamino]-1H-indole-5-carboxylic acid dimethylamide; 2-[4-(5-bromo-2,3-dihydrobenzofuran-7-yl)-2-hydroxy-4-methyl-2-trifluoromethylpentylamino]-1H-indole-5-carboxylic acid cyanomethylamide; 2-[4-(5-chloro-2,3-dihydrobenzofuran-7-yl)-2-hydroxy-4-methyl-2-trifluoromethylpentylamino]-5-methyl-1,5-dihydropyrrolo[3,2-c]pyridin-6-one; 4-(5-chloro-2,3-dihydrobenzofuran-7-yl)-1,1,1-trifluoro-2-[(6-methoxy-5,6-dihydro-1H-pyrrolo[3,2-c]pyridin-2-ylmethyl)amino]-4-methylpentan-2-ol; 2-[4-(5-chloro-2,3-dihydrobenzofuran-7-yl)-2-hydroxy-4-methyl-2-trifluoromethylpentylamino]-1,7-dihydropyrrolo[3,2-c]pyridine-4,6-dione; and 6-[4-(5-chloro-2,3-dihydrobenzofuran-7-yl)-2-hydroxy-4-methyl-2-trifluoromethylpentylamino]-3-methyl-1,7-dihydropyrrolo[2,3-d]pyrimidine-2,4-dione.

In still another embodiment, the present invention provides a method for producing a DIGRA compound having Formula Ia or Ib, wherein

(a) A is an aryl or heteroaryl group, each optionally independently substituted with one to three substituent groups, which are independently selected from the group consisting of C₁-C₅ alkyl, C₂-C₅ alkenyl, C₂-C₅ alkynyl, C₁-C₃ alkanoyl, C₃-C₈ cycloalkyl, heterocyclyl, aryl, heteroaryl, C₁-C₅ alkoxy, C₂-C₅ alkenyloxy, C₂-C₅ alkynyloxy, aryloxy, acyl, C₁-C₅ alkoxycarbonyl, aroyl, aminocarbonyl, alkylaminocarbonyl, dialkylaminocarbonyl, aminocarbonyloxy, C₁-C₅ alkylaminocarbonyloxy, C₁-C₅ dialkylaminocarbonyloxy, C₁-C₅ alkanoylamino, C₁-C₅ alkoxycarbonylamino, C₁-C₅ alkylsulfonylamino, aminosulfonyl, C₁-C₅ alkylaminosulfonyl, C₁-C₅ dialkylaminosulfonyl, halogen, hydroxy, carboxy, cyano, trifluoromethyl, trifluoromethoxy, nitro, amino wherein the nitrogen atom is optionally independently mono- or di-substituted by C₁-C₅ alkyl or aryl, ureido wherein either nitrogen atom is optionally independently substituted with C₁-C₅ alkyl, C₁-C₅ alkylthio wherein the sulfur atom is optionally oxidized to a sulfoxide or sulfone;

(b) R¹ and R² are each independently hydrogen or C₁-C₅ alkyl;

(c) R³ is hydrogen, C₁-C₈ alkyl, C₂-C₈ alkenyl, C₂-C₈ alkynyl, carbocycle, heterocyclyl, aryl, heteroaryl, carbocycle-C₁-C₈ alkyl, carboxy, alkoxycarbonyl, aryl-C₁-C₈ alkyl, aryl-C₁-C₈ haloalkyl, heterocyclyl-C₁-C₈ alkyl, heteroaryl-C₁-C₈ alkyl, carbocycle-C₂-C₈ alkenyl, aryl-C₂-C₈ alkenyl, heterocyclyl-C₂-C₈ alkenyl, or heteroaryl-C₂-C₈ alkenyl, each optionally independently substituted with one to three substituent groups, wherein each substituent group of R³ is independently C₁-C₅ alkyl, C₂-C₅ alkenyl, C₂-C₅ alkynyl, C₃-C₈ cycloalkyl, phenyl, C₁-C₅ alkoxy, phenoxy, C₁-C₅ alkanoyl, aroyl, C₁-C₅ alkoxycarbonyl, C₁-C₅ alkanoyloxy, aminocarbonyloxy, C₁-C₅ alkylaminocarbonyloxy, C₁-C₅ dialkylaminocarbonyloxy, aminocarbonyl, C₁-C₅ alkylaminocarbonyl, C₁-C₅ dialkylaminocarbonyl, C₁-C₅ alkanoylamino, C₁-C₅ alkoxycarbonylamino, C₁-C₅ alkylsulfonylamino, C₁-C₅ alkylaminosulfonyl, C₁-C₅ dialkylaminosulfonyl, halogen, hydroxy, carboxy, cyano, oxo, trifluoromethyl, nitro, amino wherein the nitrogen atom is optionally independently mono- or di-substituted by C₁-C₅ alkyl, ureido wherein either nitrogen atom is optionally independently substituted with C₁-C₅ alkyl, C₁-C₅ alkylthio wherein the sulfur atom is optionally oxidized to a sulfoxide or sulfone, wherein R³ cannot be trifluoromethyl;

(d) B is a methylene or substituted methylene group having one or two substituent groups independently selected from the group consisting of C₁-C₃ alkyl, hydroxy, halogen, amino, and oxo;

(e) D is —NH— or —NR′—, wherein R′ comprises an unsubstituted or substituted C₁-C₁₅ (alternatively, C₁-C₁₀, or C₁-C₅, or C₁-C₃) linear or branched alkyl group;

(f) E is the hydroxy group; and

(g) Q comprises a heteroaryl group optionally independently substituted with one to three substituent groups, which are independently selected from the group consisting of C₁-C₅ alkyl, C₂-C₅ alkenyl, C₂-C₅ alkynyl, C₁-C₃ alkanoyl, C₃-C₈ cycloalkyl, heterocyclyl, aryl, heteroaryl, C₁-C₅ alkoxy, C₂-C₅ alkenyloxy, C₂-C₅ alkynyloxy, aryloxy, acyl, C₁-C₅ alkoxycarbonyl, aroyl, aminocarbonyl, alkylaminocarbonyl, dialkylaminocarbonyl, aminocarbonyloxy, C₁-C₅ alkylaminocarbonyloxy, C₁-C₅ dialkylaminocarbonyloxy, C₁-C₅ alkanoylamino, C₁-C₅ alkoxycarbonylamino, C₁-C₅ alkylsulfonylamino, aminosulfonyl, C₁-C₅ alkylaminosulfonyl, C₁-C₅ dialkylaminosulfonyl, halogen, hydroxy, carboxy, cyano, trifluoromethyl, trifluoromethoxy, nitro, amino wherein the nitrogen atom is optionally independently mono- or di-substituted by C₁-C₅ alkyl or aryl, ureido wherein either nitrogen atom is optionally independently substituted with C₁-C₅ alkyl, C₁-C₅ alkylthio wherein the sulfur atom is optionally oxidized to a sulfoxide or sulfone, wherein each substituent group of Q is optionally independently substituted with one to three substituent groups selected from the group consisting of C₁-C₃ alkyl, C₁-C₃ alkoxy, acyl, C₁-C₃ silanyloxy, C₁-C₅ alkoxycarbonyl, carboxy, halogen, hydroxy, oxo, cyano, heteroaryl, heterocyclyl, amino wherein the nitrogen atom is optionally independently mono- or di-substituted by C₁-C₅ alkyl or aryl, ureido wherein either nitrogen atom is optionally independently substituted with C₁-C₅ alkyl, or trifluoromethyl.

Non-limiting examples of these compounds include 2-cyclopropyl-4-(5-fluoro-2-methoxyphenyl)-4-methyl-1-[(1H-pyrrolo[3,2-c]pyridin-2-yl)amino]pentan-2-ol; 2-cyclopropyl-4-(5-fluoro-2-methylphenyl)-4-methyl-1-[(1H-pyrrolo[2,3-c]pyridin-2-yl)amino]pentan-2-ol; 4-(5-chloro-2,3-dihydrobenzofuran-7-yl)-2-cyclopropyl-4-methyl-1-[(1H-pyrrolo[2,3-c]pyridin-2-yl)amino]pentan-2-ol; 2-cyclopropyl-4-(5-fluoro-2-methylphenyl)-4-methyl-1-[(1H-pyrrolo[3,2-c]pyridin-2-yl)amino]pentan-2-ol; 4-(5-chloro-2,3-dihydrobenzofuran-7-yl)-2-cyclopropyl-4-methyl-1-[(1H-pyrrolo[3,2-c]pyridin-2-yl)amino]pentan-2-ol; 4-(5-fluoro-2-methoxyphenyl)-2,4-dimethyl-1-[(1H-pyrrolo[2,3-c]pyridin-2-yl)amino]pentan-2-ol; 2-cyclohexyl-4-(5-fluoro-2-methoxyphenyl)-4-methyl-1-[(1H-pyrrolo[2,3-c]pyridin-2-yl)amino]pentan-2-ol; 2-cyclopentyl-4-(5-fluoro-2-methoxyphenyl)-4-methyl-1-[(1H-pyrrolo[2,3-c]pyridin-2-yl)amino]pentan-2-ol; 1,1-difluoro-4-(5-fluoro-2-methoxyphenyl)-4-methyl-2-[(1H-pyrrolo[2,3-c]pyridin-2-ylmethyl)amino]pentan-2-ol; 2-cyclobutyl-4-(5-fluoro-2-methoxyphenyl)-4-methyl-1-[(1H-pyrrolo[2,3-c]pyridin-2-yl)amino]pentan-2-ol; 1-fluoro-4-(5-fluoro-2-methoxyphenyl)-4-methyl-2-[(1H-pyrrolo[2,3-c]pyridin-2-ylmethyl)amino]pentan-2-ol; 4-(5-fluoro-2-methoxyphenyl)-4-methyl-2-phenyl-1-[(1H-pyrrolo[2,3-c]pyridin-2-yl)amino]pentan-2-ol; 1,1-difluoro-4-(5-fluoro-2-methoxyphenyl)-4-methyl-2-[(1H-pyrrolo[3,2-c]pyridin-2-ylmethyl)amino]pentan-2-ol; 2-(1-fluorocyclopropyl)-4-(5-fluoro-2-methoxyphenyl)-4-methyl-1-[(1H-pyrrolo[2,3-c]pyridin-2-yl)amino]pentan-2-ol; 2-(1-fluorocyclopropyl)-4-(4-fluorophenyl)-4-methyl-(1-quinolin-4-ylamino)pentan-2-ol; 4-(5-chloro-2,3-dihydrobenzofuran-7-yl)-1,1-difluoro-4-methyl-2-[(1H-pyrrolo[3,2-c]pyridin-2-ylmethyl)amino]pentan-2-ol; 4-(5-chloro-2,3-dihydrobenzofuran-7-yl)-1,1-difluoro-4-methyl-2-[(pyrrolo[3,2-b]pyridin-1-ylmethyl)amino]pentan-2-ol; 4-(5-fluoro-2-methylphenyl)-2,4-dimethyl-1-[(5-phenyl-1H-pyrrolo[2,3-c]pyridin-2-yl)amino]pentan-2-ol; 4-(5-chloro-2,3-dihydrobenzofuran-7-yl)-1,1-difluoro-4-methyl-2-[(6-methyl-1H-pyrrolo[3,2-c]pyridin-2-ylmethyl)amino]pentan-2-ol; 4-(5-chloro-2,3-dihydrobenzofuran-7-yl)-2,4-dimethyl-1-[(5-phenyl-1H-pyrrolo[2,3-c]pyridin-2-yl)amino]pentan-2-ol; 1,1-difluoro-4-(5-methanesulfonyl-2,3-dihydrobenzofuran-7-yl)-4-methyl-2-[(1H-pyrrolo[2,3-c]pyridin-2-ylmethyl)amino]pentan-2-ol; and 2-(5-bromo-1H-indol-2-ylmethyl)amino-1,1-difluoro-4-(5-methanesulfonyl-2,3-dihydrobenzofuran-7-yl)-4-methylpentan-2-ol.

In still another embodiment, the present invention provides a method for producing a DIGRA compound having Formula Ia or Ib, wherein

(a) A is an aryl or heteroaryl group, each optionally independently substituted with one to three substituent groups, which are independently selected from the group consisting of C₁-C₅ alkyl, C₂-C₅ alkenyl, C₂-C₅ alkynyl, C₁-C₃ alkanoyl, C₃-C₈ cycloalkyl, heterocyclyl, aryl, heteroaryl, C₁-C₅ alkoxy, C₂-C₅ alkenyloxy, C₂-C₅ alkynyloxy, aryloxy, acyl, C₁-C₅ alkoxycarbonyl, aroyl, aminocarbonyl, alkylaminocarbonyl, dialkylaminocarbonyl, aminocarbonyloxy, C₁-C₅ alkylaminocarbonyloxy, C₁-C₅ dialkylaminocarbonyloxy, C₁-C₅ alkanoylamino, C₁-C₅ alkoxycarbonylamino, C₁-C₅ alkylsulfonylamino, aminosulfonyl, C₁-C₅ alkylaminosulfonyl, C₁-C₅ dialkylaminosulfonyl, halogen, hydroxy, carboxy, cyano, trifluoromethyl, trifluoromethoxy, nitro, amino wherein the nitrogen atom is optionally independently mono- or di-substituted by C₁-C₅ alkyl or aryl, ureido wherein either nitrogen atom is optionally independently substituted with C₁-C₅ alkyl, C₁-C₅ alkylthio wherein the sulfur atom is optionally oxidized to a sulfoxide or sulfone;

(b) R¹ and R² are each independently C₁-C₅ alkyl, wherein one or both are independently substituted with hydroxy, C₁-C₅ alkoxy, C₁-C₅ alkylthio wherein the sulfur atom is optionally oxidized to a sulfoxide or sulfone, amino wherein the nitrogen atom is optionally independently mono- or di-substituted by C₁-C₅ alkyl or aryl;

(c) R³ is hydrogen, C₁-C₈ alkyl (preferably C₁-C₅ alkyl, more preferably C₁-C₃ alkyl), C₂-C₈ alkenyl (preferably C₁-C₅ alkenyl, more preferably C₁-C₃ alkenyl), C₂-C₈ alkynyl (preferably C₁-C₅ alkynyl, more preferably C₁-C₃ alkynyl), carbocycle, heterocyclyl, aryl, heteroaryl, carbocycle-C₁-C₈ alkyl, carboxy, alkoxycarbonyl, aryl-C₁-C₈ alkyl, aryl-C₁-C₈ haloalkyl, heterocyclyl-C₁-C₈ alkyl, heteroaryl-C₁-C₈ alkyl, carbocycle-C₂-C₈ alkenyl, aryl-C₂-C₈ alkenyl, heterocyclyl-C₂-C₈ alkenyl, or heteroaryl-C₂-C₈ alkenyl, each optionally independently substituted with one to three substituent groups, wherein each substituent group of R³ is independently C₁-C₅ alkyl, C₂-C₅ alkenyl, C₂-C₅ alkynyl, C₃-C₈ cycloalkyl, phenyl, C₁-C₅ alkoxy, phenoxy, C₁-C₅ alkanoyl, aroyl, C₁-C₅ alkoxycarbonyl, C₁-C₅ alkanoyloxy, aminocarbonyloxy, C₁-C₅ alkylaminocarbonyloxy, C₁-C₅ dialkylaminocarbonyloxy, aminocarbonyl, C₁-C₅ alkylaminocarbonyl, C₁-C₅ dialkylaminocarbonyl, C₁-C₅ alkanoylamino, C₁-C₅ alkoxycarbonylamino, C₁-C₅ alkylsulfonylamino, C₁-C₅ alkylaminosulfonyl, C₁-C₅ dialkylaminosulfonyl, halogen, hydroxy, carboxy, cyano, oxo, trifluoromethyl, nitro, amino wherein the nitrogen atom is optionally independently mono- or di-substituted by C₁-C₅ alkyl, ureido wherein either nitrogen atom is optionally independently substituted with C₁-C₅ alkyl, C₁-C₅ alkylthio wherein the sulfur atom is optionally oxidized to a sulfoxide or sulfone;

(d) B is methylene;

(e) D is —NH— group;

(f) E is the hydroxy group; and

(g) Q comprises a heteroaryl group optionally independently substituted with one to three substituent groups, which are independently selected from the group consisting of C₁-C₅ alkyl, C₂-C₅ alkenyl, C₂-C₅ alkynyl, C₁-C₃ alkanoyl, C₃-C₈ cycloalkyl, heterocyclyl, aryl, heteroaryl, C₁-C₅ alkoxy, C₂-C₅ alkenyloxy, C₂-C₅ alkynyloxy, aryloxy, acyl, C₁-C₅ alkoxycarbonyl, aroyl, aminocarbonyl, alkylaminocarbonyl, dialkylaminocarbonyl, aminocarbonyloxy, C₁-C₅ alkylaminocarbonyloxy, C₁-C₅ dialkylaminocarbonyloxy, C₁-C₅ alkanoylamino, C₁-C₅ alkoxycarbonylamino, C₁-C₅ alkylsulfonylamino, aminosulfonyl, C₁-C₅ alkylaminosulfonyl, C₁-C₅ dialkylaminosulfonyl, halogen, hydroxy, carboxy, cyano, trifluoromethyl, trifluoromethoxy, nitro, amino wherein the nitrogen atom is optionally independently mono- or di-substituted by C₁-C₅ alkyl or aryl, ureido wherein either nitrogen atom is optionally independently substituted with C₁-C₅ alkyl, C₁-C₅ alkylthio wherein the sulfur atom is optionally oxidized to a sulfoxide or sulfone, wherein each substituent group of Q is optionally independently substituted with one to three substituent groups selected from the group consisting of C₁-C₃ alkyl, C₁-C₃ alkoxy, acyl, C₁-C₃ silanyloxy, C₁-C₅ alkoxycarbonyl, carboxy, halogen, hydroxy, oxo, cyano, heteroaryl, heterocyclyl, amino wherein the nitrogen atom is optionally independently mono- or di-substituted by C₁-C₅ alkyl or aryl, ureido wherein either nitrogen atom is optionally independently substituted with C₁-C₅ alkyl, or trifluoromethyl.

In yet another embodiment, the present invention provides a method for producing a DIGRA compound having Formula Ia or Ib, wherein

(a) A is an aryl, heteroaryl, heterocyclyl, or C₃-C₈ cycloalkyl group, each optionally independently substituted with one to three substituent groups, which are independently selected from the group consisting of C₁-C₅ alkyl, C₂-C₅ alkenyl, C₂-C₅ alkynyl, C₁-C₃ alkanoyl, C₃-C₈ cycloalkyl, heterocyclyl, aryl, heteroaryl, C₁-C₅ alkoxy, C₂-C₅ alkenyloxy, C₂-C₅ alkynyloxy, aryloxy, acyl, C₁-C₅ alkoxycarbonyl, aroyl, aminocarbonyl, alkylaminocarbonyl, dialkylaminocarbonyl, aminocarbonyloxy, C₁-C₅ alkylaminocarbonyloxy, C₁-C₅ dialkylaminocarbonyloxy, C₁-C₅ alkanoylamino, C₁-C₅ alkoxycarbonylamino, C₁-C₅ alkylsulfonylamino, aminosulfonyl, C₁-C₅ alkylaminosulfonyl, C₁-C₅ dialkylaminosulfonyl, halogen, hydroxy, carboxy, cyano, trifluoromethyl, trifluoromethoxy, nitro, amino wherein the nitrogen atom is optionally independently mono- or di-substituted by C₁-C₅ alkyl or aryl, ureido wherein either nitrogen atom is optionally independently substituted with C₁-C₅ alkyl, C₁-C₅ alkylthio wherein the sulfur atom is optionally oxidized to a sulfoxide or sulfone;

(b) R¹ and R² are each independently hydrogen or C₁-C₅ alkyl;

(c) R³ is the trifluoromethyl group;

(d) B is C₁-C₅ alkylene, C₂-C₅ alkenylene, or C₂-C₅ alkynylene, each optionally independently substituted with one to three substituent groups, wherein each substituent group of B is independently C₁-C₃ alkyl, hydroxy, halogen, amino, or oxo;

(e) D is —NH—, —NR′—, —OC(O)—, —C(O)NH—, —C(O)N(R′)—, —C(O)—, or —S— group, wherein R′ comprises an unsubstituted or substituted C₁-C₁₅ (alternatively, C₁-C₁₀, or C₁-C₅, or C₁-C₃) linear or branched alkyl group;

(f) E is the hydroxy group; and

(g) Q comprises an indolyl group optionally substituted with one to three substituent groups, wherein each substituent group of Q is independently C₁-C₅ alkyl, C₂-C₅ alkenyl, C₂-C₅ alkynyl, C₃-C₈ cycloalkyl, heterocyclyl, aryl, heteroaryl, C₁-C₅ alkoxy, C₂-C₅ alkenyloxy, C₂-C₅ alkynyloxy, aryloxy, acyl, C₁-C₅ alkoxycarbonyl, C₁-C₅ alkanoyloxy, aminocarbonyl, alkylaminocarbonyl, dialkylaminocarbonyl, aminocarbonyloxy, C₁-C₅ alkylaminocarbonyloxy, C₁-C₅ dialkylaminocarbonyloxy, C₁-C₅ alkanoylamino, C₁-C₅ alkoxycarbonylamino, C₁-C₅ alkylsulfonylamino, aminosulfonyl, C₁-C₅ alkylaminosulfonyl, C₁-C₅ dialkylaminosulfonyl, halogen, hydroxy, carboxy, cyano, trifluoromethyl, trifluoromethoxy, trifluoromethylthio, nitro, amino wherein the nitrogen atom is optionally independently mono- or di-substituted by C₁-C₅ alkyl, ureido wherein either nitrogen atom is optionally independently substituted with C₁-C₅ alkyl, or C₁-C₅ alkylthio wherein the sulfur atom is optionally oxidized to a sulfoxide or sulfone, wherein each substituent group of Q is optionally independently substituted with one to three substituent groups selected from the group consisting of C₁-C₃ alkyl, C₁-C₃ alkoxy, halogen, hydroxy, oxo, cyano, amino, and trifluoromethyl.

Non-limiting examples of these compounds include 4-(5-bromo-2,3-dihydrobenzofuran-7-yl)-1,1,1-trifluoro-2-[(1H-indol-2-ylmethyl)amino]-4-methylpentan-2-ol; 1,1,1-trifluoro-2-(1H-indol-2-ylmethylamino)-4-methyl-4-pyridin-2-ylpentan-2-ol; 4-(2,3-dihydro-5-cyanobenzofuran-7-yl)-1,1,1-trifluoro-2-((1H-indol-2-yl-methyl)amino)]-4-methylpentan-2-ol; 4-(2,3-dihydrobenzofuran-7-yl)-1,1,1-trifluoro-2-[(1H-indol-2-ylmethyl)amino]-4-methylpentan-2-ol; 1,1,1-trifluoro-4-(5-fluoro-2,3-dihydrobenzofuran-7-yl)-2-[(1H-indol-2-ylmethyl)amino]-4-methylpentan-2-ol; 1,1,1-trifluoro-2-[(1H-indol-2-ylmethyl)amino]-4-methyl-4-(5-methyl-2,3-dihydrobenzofuran-7-yl)pentan-2-ol; 4-(2,3-dihydrobenzofuran-5-yl)-1,1,1-trifluoro-2-[(1H-indol-2-ylmethyl)amino]-4-methylpentan-2-ol; 4-(2,3-dihydrobenzofuran-7-yl)-1,1,1-trifluoro-4-methyl-2-[(5-trifluoromethyl-1H-indol-2-ylmethyl)amino]pentan-2-ol; and 1,1,1-trifluoro-2-[(1H-indol-2-ylmethyl)amino]-4-methyl-4-thiophen-3-ylpentan-2-ol.

In a further embodiment, the present invention provides a method for producing a DIGRA compound having Formula Ia or Ib, wherein

(a) A is an aryl or heteroaryl group, each optionally independently substituted with one to three substituent groups, which are independently selected from the group consisting of C₁-C₅ alkyl, C₂-C₅ alkenyl, C₂-C₅ alkynyl, C₁-C₃ alkanoyl, C₃-C₈ cycloalkyl, heterocyclyl, aryl, heteroaryl, C₁-C₅ alkoxy, C₂-C₅ alkenyloxy, C₂-C₅ alkynyloxy, aryloxy, acyl, C₁-C₅ alkoxycarbonyl, aroyl, aminocarbonyl, alkylaminocarbonyl, dialkylaminocarbonyl, aminocarbonyloxy, C₁-C₅ alkylaminocarbonyloxy, C₁-C₅ dialkylaminocarbonyloxy, C₁-C₅ alkanoylamino, C₁-C₅ alkoxycarbonylamino, C₁-C₅ alkylsulfonylamino, aminosulfonyl, C₁-C₅ alkylaminosulfonyl, C₁-C₅ dialkylaminosulfonyl, halogen, hydroxy, carboxy, cyano, trifluoromethyl, trifluoromethoxy, nitro, amino wherein the nitrogen atom is optionally independently mono- or di-substituted by C₁-C₅ alkyl or aryl, ureido wherein either nitrogen atom is optionally independently substituted with C₁-C₅ alkyl, C₁-C₅ alkylthio wherein the sulfur atom is optionally oxidized to a sulfoxide or sulfone;

(b) R¹ and R² are each independently hydrogen or C₁-C₅ alkyl, or R¹ and R² together with the carbon atom they are commonly attached to form a C₃-C₈ spiro cycloalkyl ring;

(c) R³ is carbocycle, heterocyclyl, aryl, heteroaryl, carbocycle-C₁-C₈ alkyl, carboxy, alkoxycarbonyl, aryl-C₁-C₈ alkyl, aryl-C₁-C₈ haloalkyl, heterocyclyl-C₁-C₈ alkyl, heteroaryl-C₁-C₈ alkyl, carbocycle-C₂-C₈ alkenyl, aryl-C₂-C₈ alkenyl, heterocyclyl-C₂-C₈ alkenyl, or heteroaryl-C₂-C₈ alkenyl, each optionally independently substituted with one to three substituent groups, wherein each substituent group of R³ is independently C₁-C₅ alkyl, C₂-C₅ alkenyl, C₂-C₅ alkynyl, C₃-C₈ cycloalkyl, phenyl, C₁-C₅ alkoxy, phenoxy, C₁-C₁ alkanoyl, aroyl, C₁-C₅ alkoxycarbonyl, C₁-C₅ alkanoyloxy, aminocarbonyloxy, C₁-C₅ alkylaminocarbonyloxy, C₁-C₅ dialkylaminocarbonyloxy, aminocarbonyl, C₁-C₅ alkylaminocarbonyl, C₁-C₅ dialkylaminocarbonyl, C₁-C₅ alkanoylamino, C₁-C₅ alkoxycarbonylamino, C₁-C₅ alkylsulfonylamino, C₁-C₅ alkylaminosulfonyl, C₁-C₅ dialkylaminosulfonyl, halogen, hydroxy, carboxy, cyano, oxo, trifluoromethyl, nitro, amino wherein the nitrogen atom is optionally independently mono- or di-substituted by C₁-C₅ alkyl, ureido wherein either nitrogen atom is optionally independently substituted with C₁-C₅ alkyl, C₁-C₅ alkylthio wherein the sulfur atom is optionally oxidized to a sulfoxide or sulfone;

(d) B is a methylene or substituted methylene group having one or two substituent groups selected from the group consisting of C₁-C₃ alkyl, hydroxy, halogen, amino, and oxo;

(e) D is the —C(O)NH— or —C(O)NR′— group, wherein R′ comprises an unsubstituted or substituted C₁-C₁₅ (alternatively, C₁-C₁₀, or C₁-C₅, or C₁-C₃) linear or branched alkyl group;

(f) E is the hydroxy group; and

(g) Q comprises the group

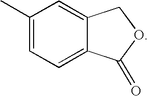

Non-limiting examples of these compounds include 3-benzyl-3-hydroxy-5-methyl-5-phenylhexanoic acid-(1-oxo-1,3-dihydroisobenzofuran-5-yl)amide; 3-hydroxy-5-methyl-3,5-diphenylhexanoic acid-(1-oxo-1,3-dihydroisobenzofuran-5-yl)amide; 3-hydroxy-5-methyl-3-phenethyl-5-phenylhexanoic acid-(1-oxo-1,3-dihydroisobenzofuran-5-yl)amide; 3-hydroxy-3-(3-methoxybenzyl)-5-methyl-5-phenylhexanoic acid-(1-oxo-1,3-dihydroisobenzofuran-5-yl)amide; 3-hydroxy-3-(4-methoxybenzyl)-5-methyl-5-phenylhexanoic acid-(1-oxo-1,3-dihydroisobenzofuran-5-yl)amide; 3-hydroxy-3-[2-(4-methoxyphenyl)ethyl]-5-methyl-5-phenylhexanoic acid-(1-oxo-1,3-dihydroisobenzofuran-5-yl)amide; 3-cyclohexylmethyl-3-hydroxy-5-methyl-5-phenylhexanoic acid-(1-oxo-1,3-dihydroisobenzofuran-5-yl)amide; 3-(4-tert-butylbenzyl)-3-hydroxy-5-methyl-5-phenylhexanoic acid-(1-oxo-1,3-dihydroisobenzofuran-5-yl)amide; 3-biphenyl-5-ylmethyl-3-hydroxy-5-methyl-5-phenylhexanoic acid-(1-oxo-1,3-dihydroisobenzofuran-5-yl)amide; 3-hydroxy-5-methyl-3-naphthalen-2-ylmethyl-5-phenylhexanoic acid-(1-oxo-1,3-dihydroisobenzofuran-5-yl)amide; 3-hydroxy-3-(3-hydroxybenzyl)-5-methyl-5-phenylhexanoic acid-(1-oxo-1,3-dihydroisobenzofuran-5-yl)amide; 3-hydroxy-5-methyl-3-(2-methyl-2-phenylpropyl)-5-phenylhexanoic acid-(1-oxo-1,3-dihydroisobenzofuran-5-yl)amide; 3-benzyl-5-(5-fluoro-2-methoxyphenyl)-3-hydroxy-5-methylhexanoic acid-(1-oxo-1,3-dihydroisobenzofuran-5-yl)amide; 3-cyclohexylmethyl-5-(5-fluoro-2-methoxyphenyl)-3-hydroxy-5-methylhexanoic acid-(1-oxo-1,3-dihydroisobenzofuran-5-yl)amide; 3-benzyl-5-(5-fluoro-2-hydroxyphenyl)-3-hydroxy-5-methylhexanoic acid-(1-oxo-1,3-dihydroisobenzofuran-5-yl)amide; 3-cyclohexylmethyl-5-(5-fluoro-2-hydroxyphenyl)-3-hydroxy-5-methylhexanoic acid-(1-oxo-1,3-dihydroisobenzofuran-5-yl)amide; 5-(5-fluoro-2-hydroxyphenyl)-3-hydroxy-5-methyl-3-(2-methyl-2-phenylpropyl)hexanoic acid-(1-oxo-1,3-dihydroisobenzofuran-5-yl)amide; 3-(2-chloro-6-fluorobenzyl)-5-(5-fluoro-2-methoxyphenyl)-3-hydroxy-5-methylhexanoic acid-(1-oxo-1,3-dihydroisobenzofuran-5-yl)amide; 3-(3-fluorobenzyl)-5-(5-fluoro-2-methoxyphenyl)-3-hydroxy-5-methylhexanoic acid-(1-oxo-1,3-dihydroisobenzofuran-5-yl)amide; 3-(2-fluorobenzyl)-5-(5-fluoro-2-methoxyphenyl)-3-hydroxy-5-methylhexanoic acid-(1-oxo-1,3-dihydroisobenzofuran-5-yl)amide; 3-(3,4-difluorobenzyl)-5-(5-fluoro-2-methoxyphenyl)-3-hydroxy-5-methylhexanoic acid-(1-oxo-1,3-dihydroisobenzofuran-5-yl)amide; 3-(2-chloro-6-fluorobenzyl)-5-(5-fluoro-2-hydroxyphenyl)-3-hydroxy-5-methylhexanoic acid-(1-oxo-1,3-dihydroisobenzofuran-5-yl)amide; 3-(3-fluorobenzyl)-5-(5-fluoro-2-hydroxyphenyl)-3-hydroxy-5-methylhexanoic acid-(1-oxo-1,3-dihydroisobenzofuran-5-yl)amide; 3-(2-fluorobenzyl)-5-(5-fluoro-2-hydroxyphenyl)-3-hydroxy-5-methylhexanoic acid-(1-oxo-1,3-dihydroisobenzofuran-5-yl)amide; 3-(3,4-difluorobenzyl)-5-(5-fluoro-2-hydroxyphenyl)-3-hydroxy-5-methylhexanoic acid-(1-oxo-1,3-dihydroisobenzofuran-5-yl)amide; 3-(4-fluorobenzyl)-5-(5-fluoro-2-methoxyphenyl)-3-hydroxy-5-methylhexanoic acid-(1-oxo-1,3-dihydroisobenzofuran-5-yl)amide; 5-(5-fluoro-2-methoxyphenyl)-3-hydroxy-5-methyl-3-(3-methylbenzyl)hexanoic acid-(1-oxo-1,3-dihydroisobenzofuran-5-yl)amide; 3-(4-fluorobenzyl)-5-(5-fluoro-2-hydroxyphenyl)-3-hydroxy-5-methylhexanoic acid-(1-oxo-1,3-dihydroisobenzofuran-5-yl)amide; 5-(5-fluoro-3-hydroxyphenyl)-3-hydroxy-5-methyl-3-(3-methylbenzyl)hexanoic acid-(1-oxo-1,3-dihydroisobenzofuran-5-yl)amide; 3-(3,5-difluorophenyl)-5-(5-fluoro-2-hydroxyphenyl)-3-hydroxy-5-methylhexanoic acid-(1-oxo-1,3-dihydroisobenzofuran-5-yl)amide; 5-(5-fluoro-2-methoxyphenyl)-3-hydroxy-5-methyl-3-(2-methylbenzyl)hexanoic acid-(1-oxo-1,3-dihydroisobenzofuran-5-yl)amide; 3-(3,5-dimethylbenzyl)-5-(5-fluoro-2-methoxyphenyl)-3-hydroxy-5-methylhexanoic acid-(1-oxo-1,3-dihydroisobenzofuran-5-yl)amide; 3-(2,5-difluorobenzyl)-5-(5-fluoro-2-methoxyphenyl)-3-hydroxy-5-methylhexanoic acid-(1-oxo-1,3-dihydroisobenzofuran-5-yl)amide; 3-(2,5-difluorobenzyl)-5-(5-fluoro-2-hydroxyphenyl)-3-hydroxy-5-methylhexanoic acid-(1-oxo-1,3-dihydroisobenzofuran-5-yl)amide; 5-(5-fluoro-2-hydroxyphenyl)-3-hydroxy-5-methyl-3-(2-methylbenzyl)hexanoic acid-(1-oxo-1,3-dihydroisobenzofuran-5-yl)amide; 3-(3,5-dimethylbenzyl)-5-(5-fluoro-2-hydroxyphenyl)-3-hydroxy-5-methylhexanoic acid-(1-oxo-1,3-dihydroisobenzofuran-5-yl)amide; 3-(3-chlorobenzyl)-5-(5-fluoro-2-hydroxyphenyl)-3-hydroxy-5-methylhexanoic acid-(1-oxo-1,3-dihydroisobenzofuran-5-yl)amide; 5-(5-fluoro-2-methoxyphenyl)-3-hydroxy-3-[2-(4-methoxyphenyl)ethyl]-5-methylhexanoic acid-(1-oxo-1,3-dihydroisobenzofuran-5-yl)amide; and 5-(5-fluoro-2-methoxyphenyl)-3-hydroxy-3-(2-methoxybenzyl)-5-methylhexanoic acid-(1-oxo-1,3-dihydroisobenzofuran-5-yl)amide. Other non-limiting examples of these compounds include 6-(2-benzyl-2-hydroxy-4-methyl-4-phenylpentylamino)isobenzofuran-1(3H)-one; 5-(2-hydroxy-4-methyl-2,4-diphenylpentylamino)isobenzofuran-1(3H)-one; 5-(2-hydroxy-4-methyl-2-phenethyl-4-phenylpentylamino)isobenzofuran-1(3H)-one; 6-(2-hydroxy-2-(3-methoxybenzyl)-4-methyl-4-phenylpentylamino)isobenzofuran-1(3H)-one; 5-(2-hydroxy-2-(4-methoxybenzyl)-4-methyl-4-phenylpentylamino)isobenzofuran-1(3H)-one; 5-(2-hydroxy-2-[2-(4-methoxyphenyl)ethyl]-4-methyl-4-phenylpentylamino)isobenzofuran-1(3H)-one; 6-(2-cyclohexylmethyl-2-hydroxy-4-methyl-4-phenylpentylamino)isobenzofuran-1(3H)-one; 5-(2-(4-tert-butylbenzyl)-2-hydroxy-4-methyl-4-phenylpentylamino)isobenzofuran-1(3H)-one; 5-(2-biphenyl-4-ylmethyl-2-hydroxy-4-methyl-4-phenylpentylamino)isobenzofuran-1(3H)-one; 5-(2-hydroxy-4-methyl-2-naphthalen-2-ylmethyl-4-phenylpentylamino)isobenzofuran-1(3H)-one; 6-(2-hydroxy-2-(3-hydroxybenzyl)-4-methyl-4-phenylpentylamino)isobenzofuran-1(3H)-one; and 5-(2-hydroxy-4-methyl-2-(2-methyl-2-phenylpropyl)-4-phenylpentylamino)isobenzofuran-1(3H)-one. Still other non-limiting examples of these compounds include (R,S)—N-(2-benzyl-2-hydroxy-4-methyl-4-phenylpentyl)-1-oxo-1,3-dihydroisobenzofuran-5-carboxamide; (R,S)—N-(2-hydroxy-4-methyl-2,4-diphenylpentyl)1-oxo-1,3-dihydroisobenzofuran-5-carboxamide; (R,S)—N-(2-hydroxy-4-methyl-2-phenethyl-4-phenylpentyl)1-oxo-1,3-dihydroisobenzofuran-6-carboxamide; (R,S)—N-(2-hydroxy-2-(3-methoxybenzyl)-4-methyl-4-phenylpentyl)-1-oxo-1,3-dihydroisobenzofuran-6-carboxamide; (R,S)—N-(2-hydroxy-2-(4-methoxybenzyl)-4-methyl-4-phenylpentyl)1-oxo-1,3-dihydroisobenzofuran-5-carboxamide; (R,S)—N-(2-hydroxy-2-[2-(4-methoxyphenyl)ethyl]-4-methyl-4-phenylpentyl)-1-oxo-1,3-dihydroisobenzofuran-6-carboxamide; (R,S)—N-(2-cyclohexylmethyl-2-hydroxy-4-methyl-4-phenylpentyl)-1-oxo-1,3-dihydroisobenzofuran-6-carboxamide; (R,S)—N-(2-(4-tert-butylbenzyl)-2-hydroxy-4-methyl-4-phenylpentyl)-1-oxo-1,3-dihydroisobenzofuran-6-carboxamide; (R,S)—N-(2-biphenyl-4-ylmethyl-2-hydroxy-4-methyl-4-phenylpentyl)-1-oxo-1,3-dihydroisobenzofuran-5-carboxamide; (R,S)—N-(2-hydroxy-4-methyl-2-naphthalen-2-ylmethyl-4-phenylpentyl)-1-oxo-1,3-dihydroisobenzofuran-5-carboxamide; (R,S)—N-(2-hydroxy-2-(3-hydroxybenzyl)-4-methyl-4-phenylpentyl)-1-oxo-1,3-dihydroisobenzofuran-6-carboxamide; and (R,S)—N-(2-hydroxy-4-methyl-2-(2-methyl-2-phenylpropyl)-4-phenylpentyl)-1-oxo-1,3-dihydroisobenzofuran-6-carboxamide.

In one aspect of the present invention, a selected stereoisomeric compound having Formula Ia or Ib is produced by a method comprising reacting a compound having Formula IVa or IVb

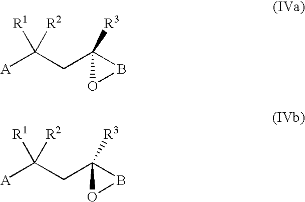

with a compound having a formula of Q-NH₂ (or Q-NHR′), Q-C(O)OH, Q-C(O)NH—R″ (or Q-C(O)N(R′)R″), or Q-SH, wherein Q has various meanings as disclosed herein above, R′ comprises an unsubstituted or substituted C₁-C₁₅ (alternatively, C₁-C₁₀, or C₁-C₅, or C₁-C₃) linear or branched alkyl group; and R″ is hydrogen or a C₁-C₅ alkyl group (preferably, C₁-C₃ alkyl group). A, B, R¹, R², and R³ have the meanings disclosed herein above.

A compound having Formula IVa or IVb can be prepared according to the method disclosed in U.S. Patent Application Publication 2005/0234250 A1, which is incorporated herein by reference.

In one embodiment, a compound having Formula Ia or Ib, wherein D is —NH— or —NR′—, is produced by reacting a compound having Formula IVa or IVb with a compound having a formula of Q-NH₂ (or Q-NHR′).

In another embodiment, a compound having Formula Ia or Ib, wherein D is —C(O)O—, is produced by reacting a compound having Formula IVa or IVb with a compound having a formula of Q-C(O)OH.

In still another embodiment, a compound having Formula Ia or Ib, wherein D is —C(O)NH— or —C(O)N(R′)—, is produced by reacting a compound having Formula IVa or IVb with a compound having a formula of Q-C(O)NHR″ or Q-C(O)N(R′)R″, wherein R″ is hydrogen or a C₁-C₅ alkyl group (preferably, C₁-C₃ alkyl group).

In still another embodiment, a compound having Formula Ia or Ib, wherein D is —S—, is produced by reacting a compound having Formula IVa or IVb with a compound having a formula of Q-SH.

In still another embodiment, a compound having Formula Ia or Ib, wherein D is —C(O)— can be produced by a method shown in Scheme 1.

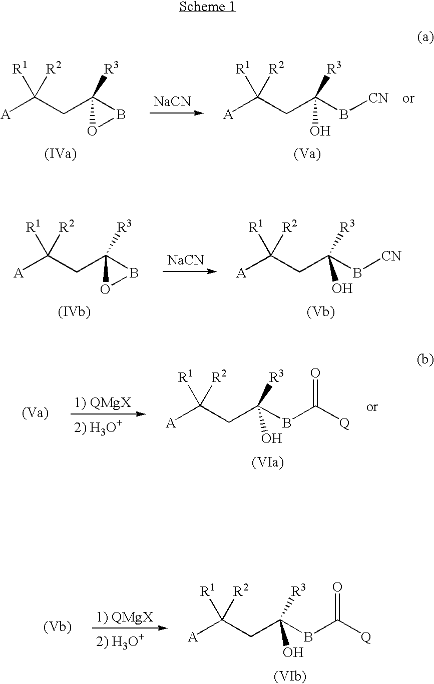

wherein X is a halogen, such as bromine, chlorine, or iodine; preferably, bromine.

In another aspect, a compound having Formula Ia can be prepared by a method as disclosed in Scheme 2.

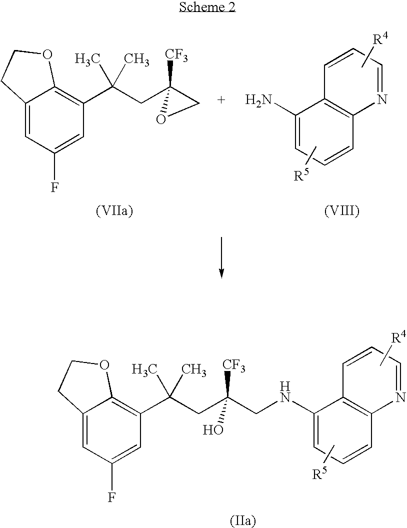

Similarly, a compound having Formula IIc can be prepared by the method of Scheme 2, wherein the aminoquinoline compound VIII is replaced by an aminoisoquinolin compound represented by

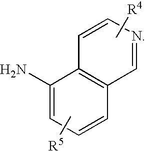

In still another aspect, a compound having Formula IIb can be prepared by a method as disclosed in Scheme 3.

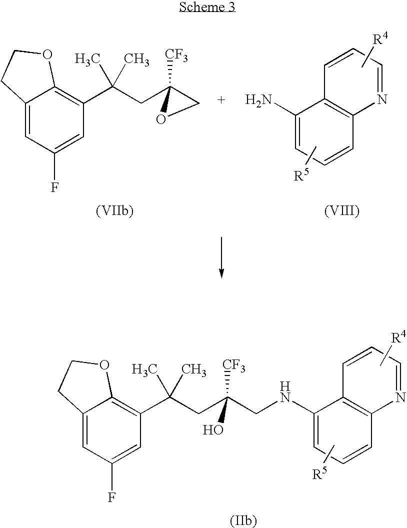

Similarly, a compound having Formula IId can be prepared by the method of Scheme 3, wherein the aminoquinoline compound VIII is replaced by an aminoisoquinolin compound represented by

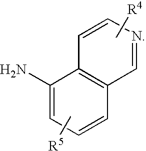

In yet another aspect, a compound having Formula IIIa can be prepared by a method as disclosed in Scheme 4.

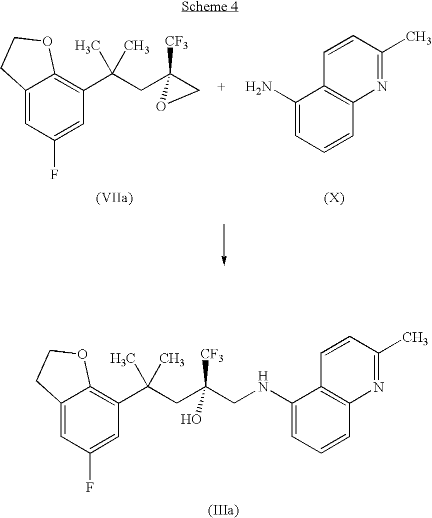

Similarly, a compound having Formula IIIc can be prepared by the method of Scheme 4, wherein the aminoquinoline compound X is replaced by an aminoisoquinolin compound represented by

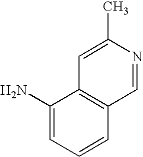

In yet another aspect, a compound having Formula IIIb can be prepared by a method as disclosed in Scheme 5.

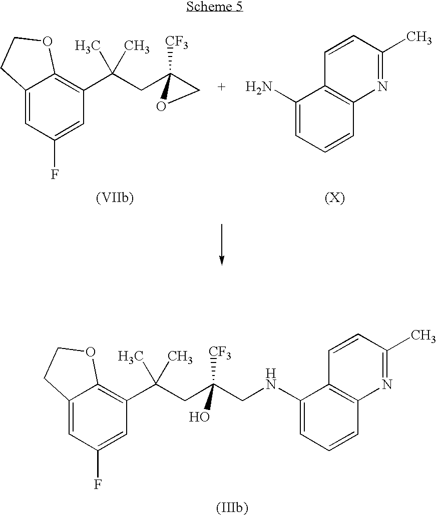

Similarly, a compound having Formula IIId can be prepared by the method of Scheme 5, wherein the aminoquinoline compound X is replaced by an aminoisoquinolin compound represented by

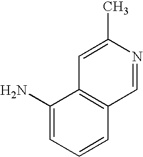

In one aspect a compound having Formula IVa or IVb can be prepared by a method that comprises:

(a) reacting a starting material of Formula XI with a chiral sulfoxide anion source XIIa or XIIb, where G is an alkyl (e.g., C₁-C₅ or C₁-C₃ alkyl) or aryl group and M is a counter-cation, in the presence of a suitable base (such as lithium diisopropylamide (“LDA”)) and a suitable solvent (such as THF) to produce a compound of Formula XIIIa or XIIIb, respectively.

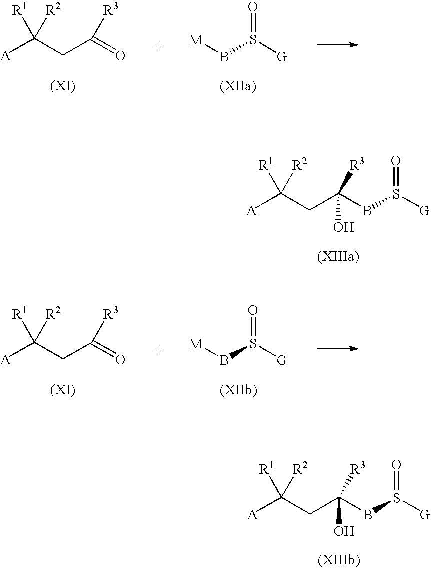

(b) reducing the sulfoxide of Formula XIIIa or XIIIb in a suitable solvent (such as THF) to obtain the compound of formula XIVa or XIVb, respectively.

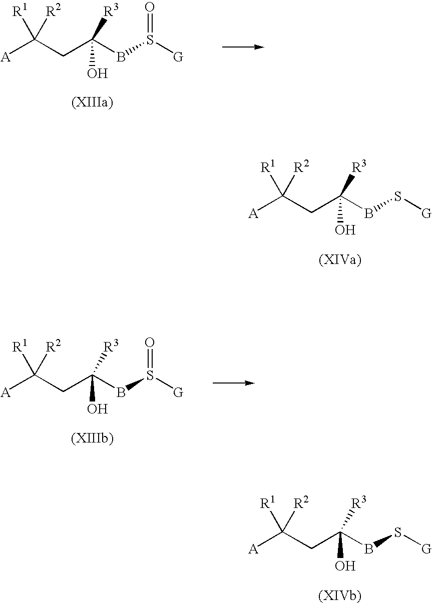

(c) cyclizing the compound of Formula XIVa or XIVb with a reagent such as trimethyloxonium tetrafluoroborate in a suitable solvent (such as dichloromethane) in the presence of a base (such as potassium carbonate) to form the epoxide compound of Formula IVa or Formula IVb, respectively.

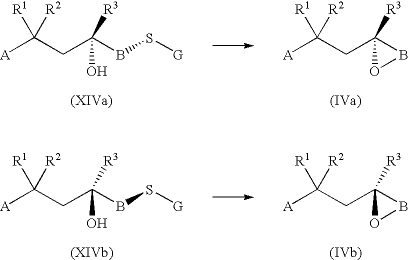

In one aspect of the invention, a suitable solvent of step (a) is diethyl ether, dipropyl ether, diisopropyl ether, dibutyl ether, tetrahydrofuran (“THF”), ethylene glycol dimethyl ether (“DME”), tert-butyl methyl ether (“MTBE”), or a mixture thereof, preferably diethyl ether or tetrahydrofuran.

In another aspect of the invention, the chiral sulfoxide anion source having Formula XIIa or XIIb is generated from the corresponding neutral sulfoxide precursor with a base selected from the group consisting of lithium diisopropylamide (“LDA”), sodium hexamethyldisilazide (“NaHMDS”), potassium hexamethyldisilazide (“KHMDS”), sodium hydride, potassium hydride, n-butyllithium, methyllithium, ethyl magnesium bromide, methylmagnesium bromide, and compatible mixtures thereof.

In still another aspect of the invention, the reduction of step (b) is accomplished using a reducing agent comprises lithium aluminum hydride (“LAH”), diisobutyl aluminum hydride (“DIBAL”), a 65% (by weight) solution of sodium bis(2-methoxyethoxy)aluminum hydride in toluene, or a mixture of trifluoroacetic acid anhydride and sodium iodide (P. Bravo et al., J. Org. Chem., Vol. 57, 2726 (1992)), a mixture of trifluoroacetic acid anhydride and 2,4,6-trimethylpyridine (P. Bravo et al., J. Org. Chem., Vol. 55, 4216 (1990)), or hydrogen chloride in ethanol (J. L. Garcia Ruano et al., J. Org. Chem., Vol. 59, 533 (1994)).

In yet another aspect of the invention, when the reduction step (b) is performed with a reducing agent, such as the aluminum hydride reagents listed above, a suitable solvent is diethyl ether, toluene, tetrahydrofuran (“THF”), tert-butyl methyl ether (“MTBE”), hexanes, or a mixture thereof. Otherwise, a suitable solvent for step (b) is diethyl ether, toluene, THF, MTBE, hexanes, benzene, acetonitrile, acetone, dichloromethane, ethyl acetate, or a mixture thereof.

In a further aspect of the invention, an alkylating agent is used in step (c), preferably an alkyl halide such as methyl iodide, methyl bromide, and ethyl iodide, or a trialkyloxonium reagent selected from trimethyloxonium tetrafluoroborate, trimethyloxonium hexachloroantimonate, triethyloxonium tetrafluoroborate, triethyloxonium hexafluorophosphate, and triethyloxonium hexachloroantimonate.

In yet another aspect of the invention, the cyclization of step (c) is accomplished with a suitable organic or inorganic base, preferably triethylamine (“TEA”), diisopropylethylamine (“DIEA”), pyridine, lutidine, sodium hydride, potassium hydride, potassium carbonate, or sodium carbonate.

In still another aspect of the invention, a suitable solvent of step (c) is dichloromethane, chloroform, dichloroethane, THF, diethyl ether, toluene, benzene, ethyl acetate, or a mixture thereof.

Optimum reaction conditions and reaction times may vary depending on the particular reactants used. Unless otherwise specified, solvents, temperatures, pressures, and other reaction conditions may be readily selected by one of ordinary skill in the art. Furthermore, if the substituent groups on R¹ to R² are incompatible under the reaction conditions of the process, protection/deprotection of these groups may be carried out, as required, using reagents and conditions readily selected by one of ordinary skill in the art (see, for example, T. W. Greene and P. G. M. Wuts, “Protective Groups in Organic Synthesis,” John Wiley & Sons, New York (1999)) and references cited therein. For example, a hydroxyl group can be protected as methyl ether and be deprotected at an appropriate stage with reagents, such as boron tribromide in dichloromethane. Typically, reaction progress may be monitored by high performance liquid chromatography (“HPLC”) or thin layer chromatography (“TLC”), if desired, and intermediates and products may be purified by chromatography on silica gel and/or by recrystallization.

In certain aspects, a compound having Formula IXa or IXb can be prepared by a method that comprises:

(a) reacting a starting material of Formula XV with a chiral sulfoxide anion source XIIa or XIIb, where G is an alkyl (e.g., C₁-C₅ or C₁-C₃ alkyl) or aryl group and M is a counter-cation, in the presence of a suitable base (such as lithium diisopropylamide (“LDA”)) and a suitable solvent (such as THF) to produce a compound of Formula XVIa or XVIb, respectively.

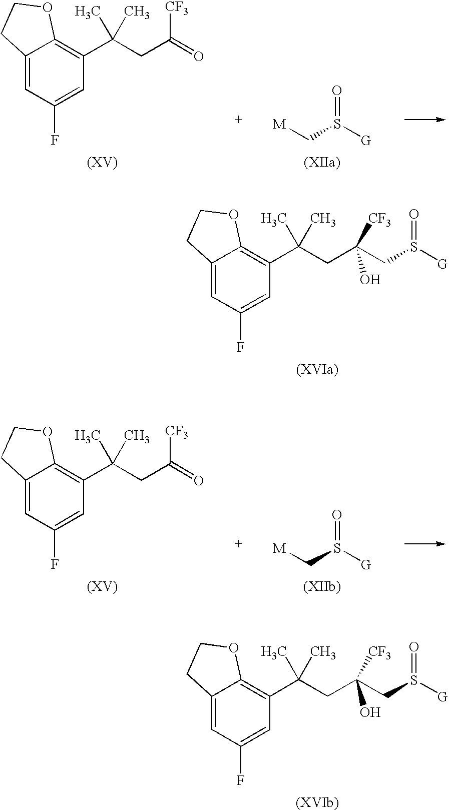

(b) reducing the sulfoxide of Formula XVIa or XVIb in a suitable solvent (such as THF) to obtain the compound of formula XVIIa or XVIIb, respectively.

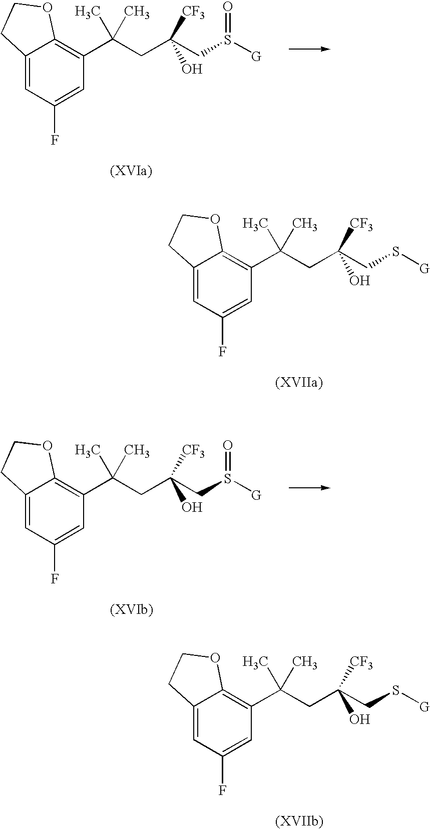

(c) cyclizing the compound of Formula XVIIa or XVIIb with a reagent such as trimethyloxonium tetrafluoroborate in a suitable solvent (such as dichloromethane) in the presence of a base (such as potassium carbonate) to form the epoxide compound of Formula VIIa or VIIb, respectively.

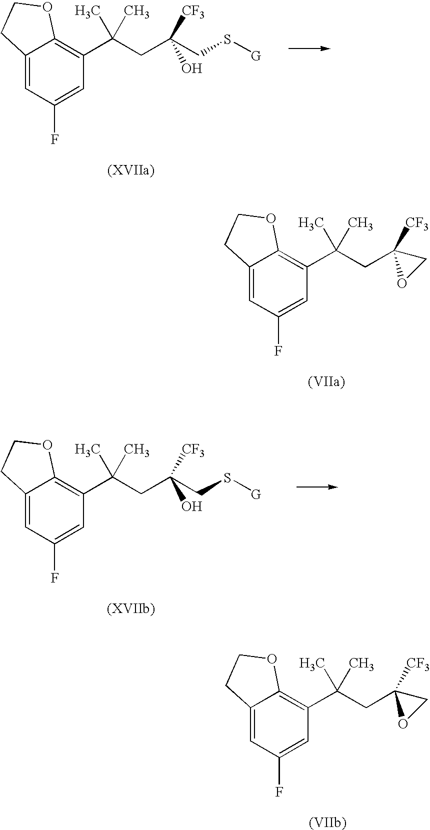

In one aspect, a compound having Formula XI can be produced by a method shown in Scheme 6 or Scheme 7.

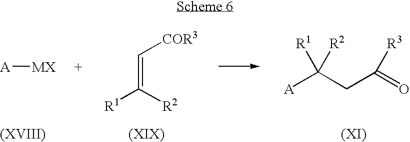

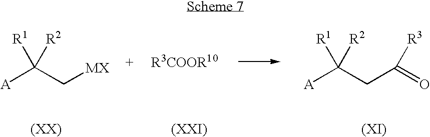

wherein M is, for example, Mg, Cu, or Li, provided that when M is Li, X is absent; X is, for example, bromine, chlorine, or iodine; R¹⁰ is a lower alkyl group (such as C₁-C₅ alkyl group; preferably, ethyl); and R¹, R², and R³ has the meanings disclosed herein above.

In one embodiment wherein A comprises a dihydrobenzofuranyl group and R¹, R², and R³ are the trifluoromethyl group, the preparation of the compound having Formula XV is as follows.

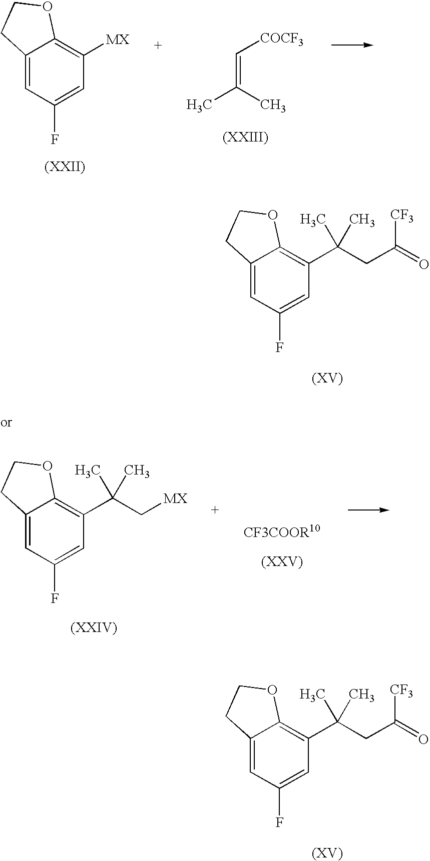

wherein M, X, and R¹⁰ have the meanings disclosed immediately above.

In another aspect, a stereoisomer having Formula Ia or Ib produced by a method of the present invention can be included in a pharmaceutical composition for treating, controlling, reducing, ameliorating, or preventing inflammation or infections and their inflammatory sequelae. In one embodiment, such a pharmaceutical composition is an ophthalmic pharmaceutical composition.

While specific embodiments of the present invention have been described in the foregoing, it will be appreciated by those skilled in the art that many equivalents, modifications, substitutions, and variations may be made thereto without departing from the spirit and scope of the invention as defined in the appended claims.

What is claimed is:

1. A method for selectively producing a stereoisomer of a substituted alcohol that has a Formula Ia or Ib, 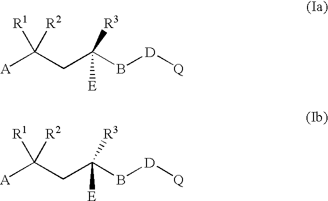
wherein A and Q are independently selected from the group consisting of unsubstituted and substituted aryl and heteroaryl groups, unsubstituted and substituted cycloalkyl and heterocycloalkyl groups, unsubstituted and substituted cycloalkenyl and heterocycloalkenyl groups, unsubstituted and substituted cycloalkynyl and heterocycloalkynyl groups, and unsubstituted and substituted heterocyclic groups; R¹ and R² are independently selected from the group consisting of hydrogen, unsubstituted C₁-C₁₅ linear or branched alkyl groups, substituted C₁-C₁₅ linear or branched alkyl groups, unsubstituted C₃-C₁₅ cycloalkyl groups, and substituted C₃-C₁₅ cycloalkyl groups; R³ is selected from the group consisting of hydrogen, unsubstituted C₁-C₁₅ linear or branched alkyl groups, substituted C₁-C₁₅ linear or branched alkyl groups, unsubstituted C₃-C₁₅ cycloalkyl and heterocycloalkyl groups, substituted C₃-C₁₅ cycloalkyl and heterocycloalkyl groups, aryl groups, heteroaryl groups, and heterocyclylic groups; B comprises a methylene or substituted methylene group, wherein one or two substituents on the methylene group are independently C₁-C₅ alkyl, hydroxy, halogen, amino, or oxo group; E is hydroxy; and D is —NH—, —NR′—, OC(O)—, C(O)NH—, C(O)N(R′)—, or —S—, wherein R′ comprises an unsubstituted or substituted C₁-C₁₅ linear or branched alkyl group; and wherein R¹ and R² together may form an unsubstituted or substituted C₃-C₁₅ cycloalkyl group; the method comprising reacting a compound having Formula IVa or IVb 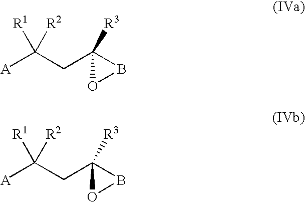
with a compound having a formula of Q-NH₂, Q-NHR′, Q-C(O)OH, Q-C(O)N—H—R″, Q-C(O)N(R′)R″, or Q-SH, wherein R″ is hydrogen or a C₁-C₅ alkyl group.

2. The method of claim 1, wherein said compound has a formula of Q-NH₂.

3. The method of claim 1, wherein said compound has a formula of Q-C(O)OH.

4. The method of claim 1, wherein said compound has a formula of Q-SH.

5. The method of claim 1, wherein A and Q are independently selected from the group consisting of unsubstituted and substituted aryl and heteroaryl groups, and unsubstituted and substituted heterocyclic groups.

6. The method of claim 1, wherein A is an unsubstituted aryl, substituted aryl, unsubstituted heteroaryl, or substituted heteroaryl group, and Q is an unsubstituted or substituted azaindolyl group.

7. The method of claim 1, wherein A is an unsubstituted aryl, substituted aryl, unsubstituted heteroaryl, or substituted heteroaryl group, and Q is a methylated benzoxazinone group.

8. The method of claim 1, wherein A is an unsubstituted aryl, substituted aryl, unsubstituted heteroaryl, or substituted heteroaryl group, and Q comprises an unsubstituted or substituted phenyl group having the formula 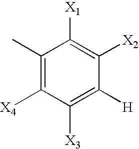
wherein X₁, X₂, X₃ and X₄ are each independently selected from the group consisting of hydrogen, halogen, hydroxy, trifluoromethyl, trifluoromethoxy, C₁-C₅ alkyl, C₂-C₅ alkenyl, C₂-C₅ alkynyl, C₁-C₅ alkoxy, C₁-C₅ alkylthio wherein the sulfur atom is optionally oxidized to a sulfoxide or sulfone, C₁-C₅ alkanoyl, C₁-C₅ alkoxycarbonyl, C₁-C₅ acyloxy, C₁-C₅ alkanoylamino, C₁-C₅ carbamoyloxy, urea, aryl, and amino wherein the nitrogen atom may be independently mono- or di-substituted by C₁-C₅ alkyl, and wherein said aryl group is optionally substituted by one or more hydroxy or C₁-C₅ alkoxy groups, and wherein either nitrogen atom of the urea group may be independently substituted by C₁-C₅ alkyl; or Q is an aromatic 5- to 7-membered monocyclic ring having from one to four heteroatoms in the ring independently selected from nitrogen, oxygen, and sulfur, optionally independently substituted with one to three substituent groups selected from the group consisting of hydrogen, halogen, hydroxy, trifluoromethyl, trifluoromethoxy, C₁-C₅ alkyl, C₂-C₅ alkenyl, C₂-C₅ alkynyl, C₁-C₅ alkoxy, C₁-C₅ alkylthio wherein the sulfur atom is optionally oxidized to a sulfoxide or sulfone, C₁-C₅ alkanoyl, C₁-C₅ alkoxycarbonyl, C₁-C₅ acyloxy, C₁-C₅ alkanoylamino, C₁-C₅ carbamoyloxy, urea, aryl optionally substituted by one or more hydroxy or C₁-C₅ alkoxy groups, and amino wherein the nitrogen atom may be independently mono- or di-substituted by C₁-C₅ alkyl, and wherein either nitrogen atom of the urea group may be independently substituted by C₁-C₅ alkyl.

9. The method of claim 1, wherein A is an unsubstituted aryl, substituted aryl, unsubstituted heteroaryl, or substituted heteroaryl group, and Q comprises an unsubstituted or substituted indolyl group with one to three substituent groups, wherein each substituent group of Q is independently C₁-C₅ alkyl, C₂-C₅ alkenyl, C₂-C₅ alkynyl, C₃-C₈ cycloalkyl, heterocyclyl, aryl, heteroaryl, C₁-C₅ alkoxy, C₂-C₅ alkenyloxy, C₂-C₅ alkynyloxy, aryloxy, acyl, C₁-C₅ alkoxycarbonyl, C₁-C₅ alkanoyloxy, aminocarbonyl, alkylaminocarbonyl, dialkylaminocarbonyl, aminocarbonyloxy, C₁-C₅ alkylaminocarbonyloxy, C₁-C₅ dialkylaminocarbonyloxy, C₁-C₅ alkanoylamino, C₁-C₅ alkoxycarbonylamino, C₁-C₅ alkylsulfonylamino, aminosulfonyl, C₁-C₅ alkylaminosulfonyl, C₁-C₅ dialkylaminosulfonyl, halogen, hydroxy, carboxy, cyano, trifluoromethyl, trifluoromethoxy, trifluoromethylthio, nitro, amino wherein the nitrogen atom is optionally independently mono- or di-substituted by C₁-C₅ alkyl, ureido wherein either nitrogen atom is optionally independently substituted with C₁-C₅ alkyl, or C₁-C₅ alkylthio wherein the sulfur atom is optionally oxidized to a sulfoxide or sulfone, wherein each substituent group of Q is unsubstituted or independently substituted with one to three substituent groups selected from the group consisting of C₁-C₃ alkyl, C₁-C₃ alkoxy, halogen, hydroxy, oxo, cyano, amino, and trifluoromethyl.

10. The method of claim 1, wherein A is an unsubstituted aryl, substituted aryl, unsubstituted heteroaryl, or substituted heteroaryl group; D is the C(O)NH— or —C(O)NR′— group, wherein R′ comprises an unsubstituted or substituted C₁-C₁₅ linear or branched alkyl group; E is the hydroxy group; and Q comprises the group

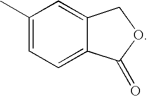

11. A method for selectively producing a stereoisomer of a substituted alcohol that has a Formula IIa or IIb, 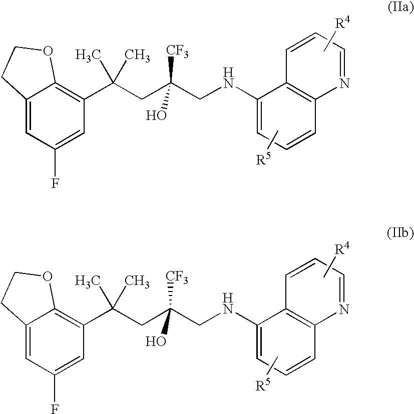
wherein R⁴ and R⁵ are independently selected from the group consisting of hydrogen, halogen, cyano, hydroxy, C₁-C₁₀ (alternatively, C₁-C₅ or C₁-C₃) alkoxy groups, unsubstituted C₁-C₁₀ (alternatively, C₁-C₅ or C₁-C₃) linear or branched alkyl groups, substituted C₁-C₁₀ (alternatively, C₁-C₅ or C₁-C₃) linear or branched alkyl groups, unsubstituted C₃-C₁₀ (alternatively, C₃-C₆ or C₃-C₅) cyclic alkyl groups, and substituted C₃-C₁₀ (alternatively, C₃-C₆ or C₃-C₅) cyclic alkyl groups; the method comprising reacting a stereoisomeric substituted dihydrobenzofuran having Formula VIIa or VIIb with a substituted quinoline having Formula VIII

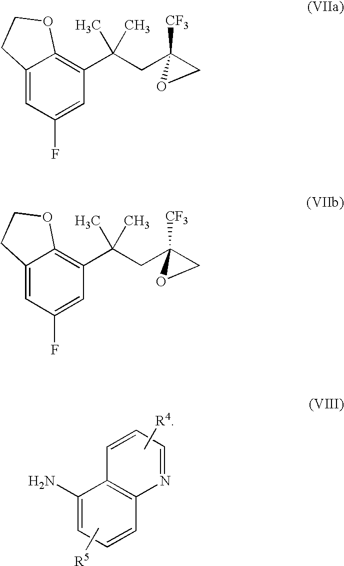

12. A method for selectively producing a stereoisomer of a substituted alcohol that has a Formula IIIa or IIIb, 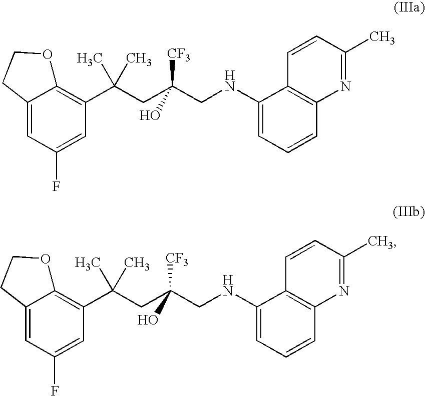
the method comprising reacting a stereoisomeric substituted dihydrobenzofuran having Formula VIIa or VIIb with a substituted quinoline having Formula X

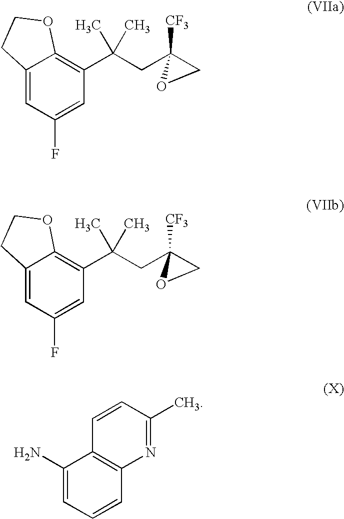

13. The method of claim 11, wherein the stereoisomeric substituted dihydrobenzofuran having Formula VIIa or VIIb is produced by a method that comprises:
(a) reacting a starting material of Formula XV with a chiral sulfoxide anion source XIIa or XIIb, where G is an alkyl (e.g., C₁-C₅ or C₁-C₃ alkyl) or aryl group and M is a counter-cation, in the presence of a suitable base and a suitable solvent to produce a compound of Formula XVIa or XVIb, respectively 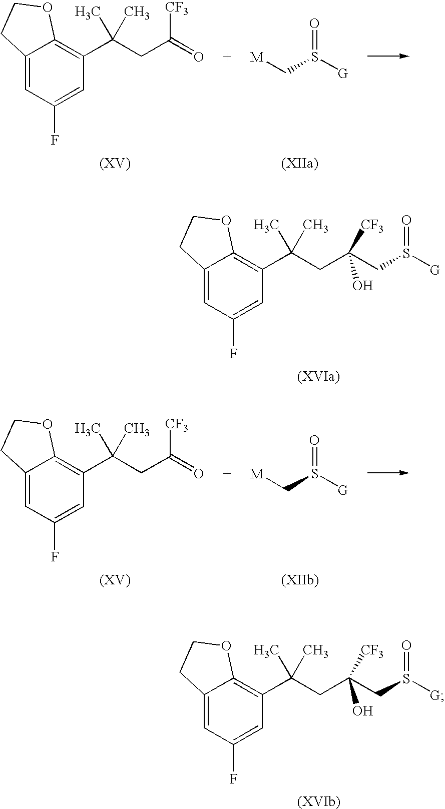
(b) reducing the sulfoxide of Formula XVIa or XVIb in a suitable to obtain the compound of formula XVIIa or XVIIb, respectively. 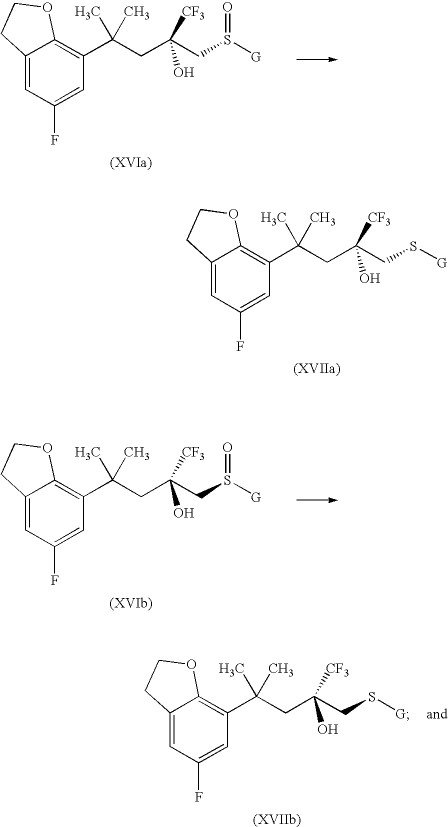
(c) cyclizing the compound of Formula XVIIa or XVIIb with a reagent such as trimethyloxonium tetrafluoroborate in a suitable solvent (such as dichloromethane) in the presence of a base (such as potassium carbonate) to form the epoxide compound of Formula VIIa or VIIb, respectively.

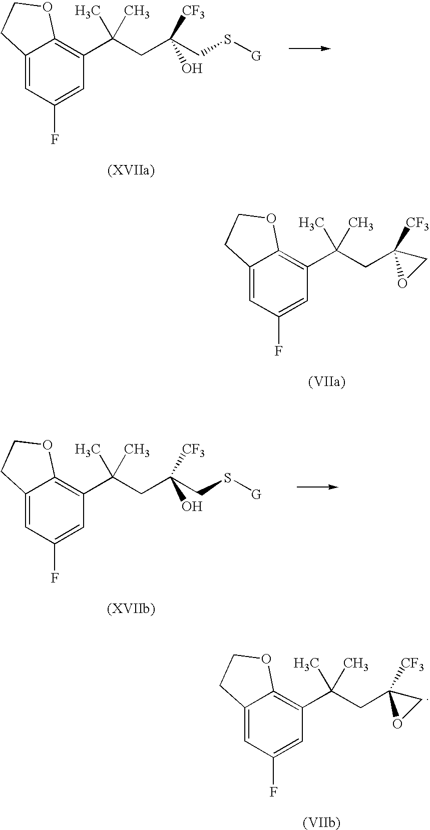

14. The method of claim 12, wherein the stereoisomeric substituted dihydrobenzofuran having Formula VIIa or VIIb is produced by a method that comprises:
(a) reacting a starting material of Formula XV with a chiral sulfoxide anion source XIIa or XIIb, where G is an alkyl (e.g., C₁-C₅ or C₁-C₃ alkyl) or aryl group and M is a counter-cation, in the presence of a suitable base and a suitable solvent to produce a compound of Formula XVIa or XVIb, respectively 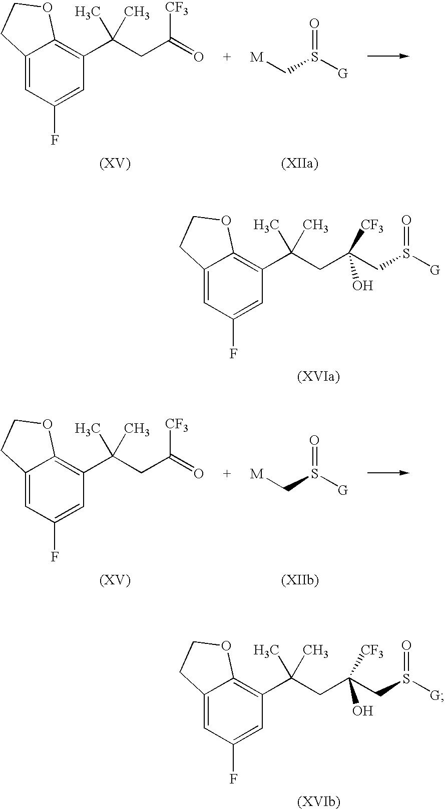
(b) reducing the sulfoxide of Formula XVIa or XVIb in a suitable solvent to obtain the compound of formula XVIIa or XVIIb, respectively. 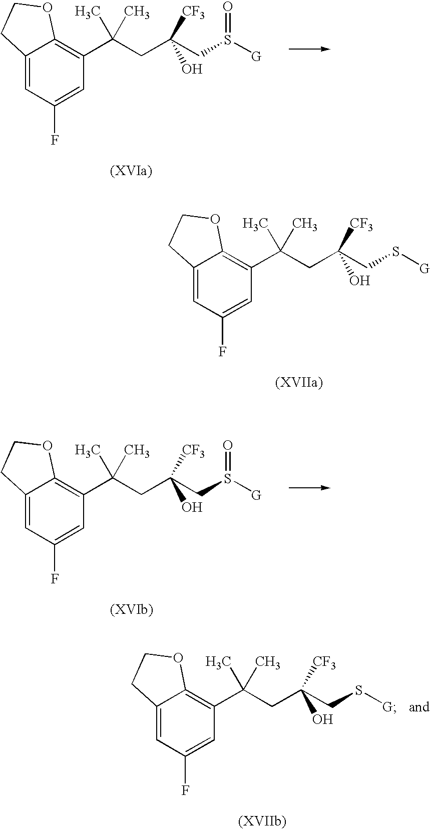
(c) cyclizing the compound of Formula XVIIa or XVIIb with a reagent such as trimethyloxonium tetrafluoroborate in a suitable solvent (such as dichloromethane) in the presence of a base (such as potassium carbonate) to form the epoxide compound of Formula VIIa or VIIb, respectively.

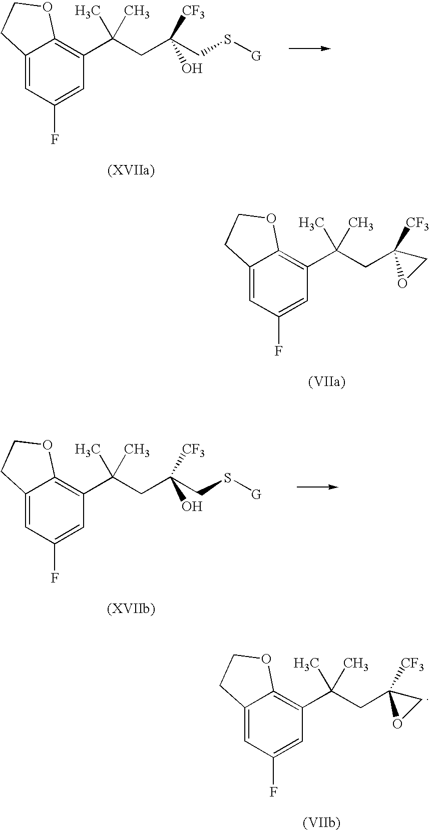

15. A method for selectively producing a stereoisomer of a substituted alcohol that has a Formula Ia or Ib, 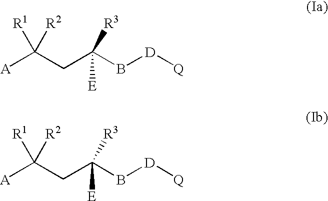
wherein A and Q are independently selected from the group consisting of unsubstituted and substituted aryl and heteroaryl groups, unsubstituted and substituted cycloalkyl and heterocycloalkyl groups, unsubstituted and substituted cycloalkenyl and heterocycloalkenyl groups, unsubstituted and substituted cycloalkynyl and heterocycloalkynyl groups, and unsubstituted and substituted heterocyclic groups; R¹ and R² are independently selected from the group consisting of hydrogen, unsubstituted C₁-C₁₅ linear or branched alkyl groups, substituted C₁-C₁₅ linear or branched alkyl groups, unsubstituted C₃-C₁₅ cycloalkyl groups, and substituted C₃-C₁₅ cycloalkyl groups; R³ is selected from the group consisting of hydrogen, unsubstituted C₁-C₁₅ linear or branched alkyl groups, substituted C₁-C₁₅ linear or branched alkyl groups, unsubstituted C₃-C₁₅ cycloalkyl and heterocycloalkyl groups, substituted C₃-C₁₅ cycloalkyl and heterocycloalkyl groups, aryl groups, heteroaryl groups, and heterocyclylic groups; B comprises a methylene or substituted methylene group, wherein one or two substituents on the methylene group are independently C₁-C₅ alkyl, hydroxy, halogen, amino, or oxo group; E is hydroxy; and D is C(O), wherein R′ comprises an unsubstituted or substituted C₁-C₁₅ linear or branched alkyl group; and wherein R¹ and R² together may form an unsubstituted or substituted C₃-C₁₅ cycloalkyl group; the method comprising:
(a) reacting a compound having Formula IVa or IVb 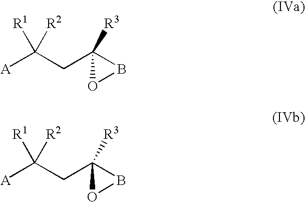
with a cyanide compound to produce an intermediate cyanide compound having a Formula XVIIIa or XVIIIb 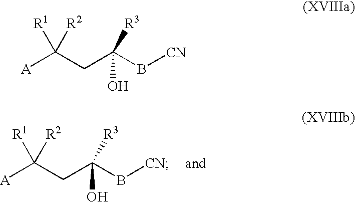
(b) reacting the intermediate cyanide compound having Formula XVIIIa or XVIIIb with a compound having a formula of Q-MgX in a presence of an acid, wherein X is a halogen.

16. The method of claim 15, wherein A comprises a 5-fluoro-2,3-dihydrobenzofuran-7-yl group.

17. The method of claim 1, wherein A comprises a 5-fluoro-2,3-dihydrobenzofuran-7-yl group.

18. A method for selectively producing a stereoisomer of a substituted alcohol that has a Formula IIc or IId, 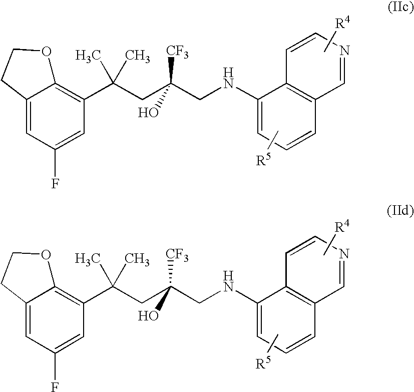
wherein R⁴ and R⁵ are independently selected from the group consisting of hydrogen, halogen, cyano, hydroxy, C₁-C₁₀ (alternatively, C₁-C₅ or C₁-C₃) alkoxy groups, unsubstituted C₁-C₁₀ (alternatively, C₁-C₅ or C₁-C₃) linear or branched alkyl groups, substituted C₁-C₁₀ (alternatively, C₁-C₅ or C₁-C₃) linear or branched alkyl groups, unsubstituted C₃-C₁₀ (alternatively, C₃-C₆ or C₃-C₅) cyclic alkyl groups, and substituted C₃-C₁₀ (alternatively, C₃-C₆ or C₃-C₅) cyclic alkyl groups; the method comprising reacting a stereoisomeric substituted dihydrobenzofuran having Formula VIIa or VIIb with a substituted isoquinoline having Formula XXVI

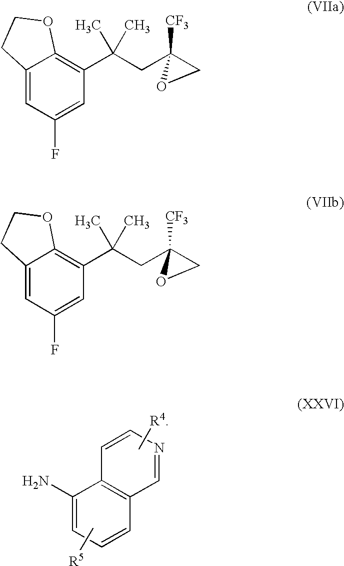

19. A single stereoisomer having Formula Ia or Ib, produced by the method of claim 1, wherein the stereoisomer is substantially free of the other stereoisomer.

20. A single stereoisomer having Formula Ia or Ib, produced by the method of claim 15, wherein the stereoisomer is substantially free of the other stereoisomer.

21. A single stereoisomer having Formula Ia or IIb, produced by the method of claim 11, wherein the stereoisomer is substantially free of the other stereoisomer.

22. A single stereoisomer having Formula IIc or IId, produced by the method of claim 18, wherein the stereoisomer is substantially free of the other stereoisomer.

23. A single stereoisomer having Formula IIIa or IIIb, produced by the method of claim 19, wherein the stereoisomer is substantially free of the other stereoisomer.
